(19) United States
(12) Patent Application Publication
(10) Pub. No.: US 2026/0235504 A1
(43) Pub. Date: Aug. 13, 2026

(54) CHEMOMETRIC MODEL SELECTION BY IMAGE ANALYSIS

(71) Applicant: TRINAMIX GMBH, Ludwigshafen am Rhein (DE)

(72) Inventors: Florian PROELL, Ludwigshafen am Rhein (DE); Michael HANKE, Ludwigshafen am Rhein (DE)

(21) Appl. No.: 19/150,606

(22) PCT Filed: Feb. 13, 2024
(86) PCT No.: PCT/EP24/53556

§ 371 Date: Jul. 24, 2025

(30) Foreign Application Priority Data
Feb. 14, 2023 (EP) 23156476.6

(51) Int. Cl. G01N 21/3563; G01N 21/01; G01N 21/31; G01N 21/359; G06V 20/68; G06V 40/10

(52) U.S. Cl. CPC G01N 21/3563; G01N 21/01; G01N 21/359; G06V 20/68; G06V 40/107; G01N 2021/3129; G01N 2201/1293; G01N 2201/1296

(57) ABSTRACT

Disclosed herein is a method of obtaining chemical composition information of at least one sample by spectroscopic measurement. The method includes the following steps: i. acquiring spectroscopic data of the sample by using at least one spectrometer device, within at least one spatial measurement range of the spectrometer device; ii. acquiring, by using at least one imaging device, image data of a scene within a field of view of the imaging device and analyzing the image data and/or the spectroscopic data thereby determining at least one item of identification information on the sample by using at least one processing device; and iii. evaluating the spectroscopic data for obtaining at least one item of sample information by using the processing device.

### TECHNICAL FIELD

The invention relates to a method of obtaining chemical composition information of at least one sample by spectroscopic measurement, a system for obtaining chemical composition information of at least one sample by spectroscopic measurement. The invention further relates to a computer program, a computer-readable storage medium and to a non-transient computer-readable medium. The method and devices can, in particular, be used for acquiring chemical information, specifically information on a chemical composition, of the sample and may in particular be used for the analysis of inhomogeneous samples.

### BACKGROUND ART

Spectrographic methods are widely used in research, industry and customer applications, enabling multiple applications such as optical analysis and/or quality control. Use cases can be found, for example, in the fields of food production and quality control, farming, pharma, medical applications, life sciences and many more. Various methods are available, such as photometry, absorption, fluorescence and Raman spectrometry, enabling qualitative and/or quantitative sample analysis. These methods usually involve acquiring spectroscopic data of a sample, also referred to as sample, by using at least one spectrometer device, which may in particular comprise at least one wavelength-selective element and at least one detector device.

Spectroscopic methods, such as near-infrared (NIR) spectroscopy, and chemometric methods may in particular be applied to obtain the chemical composition of the sample. Such samples may, in particular, be inhomogeneous samples, whose chemical composition may strongly de-pend on the exact position within the sample. Examples of inhomogeneous samples may comprise food items, e.g. fruits and/or vegetables.

In order to determine the chemical composition of a good with IR spectroscopy, an IR spectrum of a representative sample may be recorded and processed with a chemometric model. The chemometric model may translate the spectral information into a chemical composition information. Exemplary chemometric model are described in Celio Pasquini, “Near Infrared Spectroscopy: Fundamentals, Practical Aspects and Analytical Applications”, J. Braz. Chem. Soc., Vol. 14, No. 2, 198-219, 2003.

Usually, every kind of sample requires a separate chemometric model. For example, the determination of starch in wheat and corn require different chemometric models. Hence, for a measurement, a user has to select which kind of sample is measured so that an adequate chemometric model can be selected. Such an approach may be suitable for homogeneous samples like flower or oil. It would be, nevertheless, more comfortable for a user to not have to select the chemometric model or at least to allow for selecting from a pre-filtered list only, if many chemometric models are available.

However, for inhomogeneous samples the situation is even more challenging. The sample can contain chunks, for example wheat grains, which are somewhat different to each other. The situation becomes even more difficult if granular mixtures such as mix corn silage with concentrated feed are measured. The spectra of both species are superimposed, and no chemometric model is available for the particular mixture.

Xu Junli et al. “Combining deep learning with chemometrics when it is really needed: A case of real time object detection and spectral model application for spectral image processing”, ANA-LYTICA CHIMICA ACTA, ELSEVIER, AMSTERDAM, NL, vol. 1202, XP087004785, ISSN: 0003-2670, DOI: 10.1016/J.ACA.2022.339668 describes that deep learning (DL) is still in its early stage in chemometric domain for spectral image processing. A combination of DL and chemometrics is described to process spectral images even with as few as <100 spectral images.

US 2013/080070A1 describes systems and methods for identifying and selecting a more accurate chemometric model for the analysis of specific plant samples via near infrared spectrometry.

Zhu Hongyan et al. “Hyperspectral Imaging for Predicting the Internal Quality of Kiwifruits Based on Variable Selection Algorithms and Chemometric Models”, Scientific Reports, vol. 7, no. 1, 10 Aug. 2017, XP093061375, DOI: 10.1038/s41598-017-08509-6, https://www.nature.com/articles/s41598-017-08509-6 describes the feasibility and potentiality of determining firmness, soluble solids content (SSC), and pH in kiwifruits using hyperspectral imaging, combined with variable selection methods and calibration models. The images were acquired by a push-broom hyperspectral reflectance imaging system covering two spectral ranges. Weighted regression coefficients (BW), successive projections algorithm (SPA) and genetic algorithm-partial least square (GAPLS) were compared and evaluated for the selection of effective wavelengths. Moreover, multiple linear regression (MLR), partial least squares regression and least squares support vector machine (LS-SVM) are described to predict quality attributes quantitatively using effective wavelengths.

Rahman Anisur et al. “Hyperspectral imaging for predicting the allicin and soluble solid content of garlic with variable selection algorithms and chemometric models: Predicting the allicin and soluble solid content of garlic”, JOURNAL OF THE SCIENCE OF FOOD AND AGRICULTURE, vol. 98, no. 12, 14 May 2018, pages 4715-4725, XP093061381, GB, ISSN: 0022-5142, DOI: 10.1002/jsfa.9006, https://api.wiley.com/onlinelibrary/tdm/v1/articles/10.1002%2Fjsfa.9006 describes hyperspectral images of 100 garlic cloves were acquired that covered two spectral ranges, from which the mean spectra of each clove were extracted. The calibration models included partial least squares (PLS) and least squares-support vector machine (LS-SVM) regression, as well as different spectral pre-processing techniques, from which the highest performing spectral preprocessing technique and spectral range were selected. Variable selection methods, such as regression coefficients, variable importance in projection (VIP) and the successive projections algorithm (SPA), were evaluated for the selection of effective wavelengths (EWs). Furthermore, PLS and LS-SVM regression methods were applied to quantitatively predict the quality attributes of garlic using the selected EWs.

### Problem to Be Solved

It is therefore desirable to provide a means and methods, which address the above-mentioned technical challenges in the field of spectroscopic sample analysis. Specifically, means and methods shall be provided which allow for obtaining accurate spectroscopic data of a sample by taking into account possible local variations and inhomogeneity of the sample.

### SUMMARY

This problem is addressed by a method of obtaining chemical composition information of at least one sample by spectroscopic measurement, a system for obtaining chemical composition information of at least one sample by spectroscopic measurement, a computer program and a computer-readable storage medium, with the features of the independent claims. Advantageous embodiments which might be realized in an isolated fashion or in any arbitrary combinations are listed in the dependent claims as well as throughout the specification.

In a first aspect of the present invention, a method for obtaining chemical composition information of at least one sample by spectroscopic measurement is disclosed. The method comprises the following method steps, which specifically may be performed in the given order. However, a different order is also possible. The method may further comprise additional method steps, which are not listed. Further, one or more or even all of the method steps may be performed only once or repeatedly.

The method comprises the following steps:
i. acquiring spectroscopic data of the sample by using at least one spectrometer device, within at least one spatial measurement range of the spectrometer device;
ii. acquiring, by using at least one imaging device, image data of a scene within a field of view of the imaging device, the scene comprising at least a part of the sample and at least a part of the spatial measurement range of the spectrometer device, and analyzing the image data and/or the spectroscopic data thereby determining at least one item of identification information on the sample by using at least one processing device;
iii. evaluating the spectroscopic data for obtaining at least one item of sample information by using the processing device, wherein the evaluating comprises processing the spectroscopic data with at least one chemometric model, wherein the chemometric model translates the spectroscopic data into chemical composition information, wherein the chemometric model is selected in accordance with the item of identification information.

The term “spectroscopic measurement” as used herein is a broad term and is to be given its ordinary and customary meaning to a person of ordinary skill in the art and is not to be limited to a special or customized meaning. The term specifically may refer, without limitation, to acquiring spectroscopic data on at least one sample. The spectroscopic data may specifically be acquired by using at least one spectrometer device. As part of the spectroscopic measurement, the sample may be illuminated with electromagnetic radiation in the infrared spectral range, specifically in the near infrared spectral range. In particular, the electromagnetic radiation may be in a wavelength range from 760 nm to 1000 μm, specifically in a wavelength range from 760 nm to 15 μm, more specifically in a wavelength range from 1 μm to 5 μm, more specifically in a wavelength range from 1 μm to 3 μm. The electromagnetic radiation may also be referred to as light, such that these two terms are be used interchangeably in this document. The spectroscopic measurement may further comprise receiving incident light after interaction with the sample and generating at least one corresponding signal, which may form part of the spectroscopic data. The spectroscopic data may comprise information on at least one optical property or optically measurable property of the sample, which is determined as a function of the wavelength, for one or more different wavelengths. More specifically, the spectroscopic data may relate to at least one property characterizing at least one of a transmission, an absorption, a reflection and an emission of the sample. The at least one optical property, may be determined for one or more wavelengths. The spectroscopic data may specifically take the form of a signal intensity determined as a function of the wavelength of the spectrum or a partition thereof, such as a wavelength interval, wherein the signal intensity may preferably be provided as an electrical signal, which may be used for further evaluation. Thus, the spectroscopic data may be generated as part of the spectroscopic measurement.

The present invention proposes in addition to the spectroscopic measurement using image data. The combining of the spectroscopic measurement and image data may allow for obtaining accurate chemical composition information of the sample, in particular even in case of mixtures, local variations and inhomogeneity of the sample.

The term “acquiring spectroscopic data” as used herein is a broad term and is to be given its ordinary and customary meaning to a person of ordinary skill in the art and is not to be limited to a special or customized meaning. The term specifically may refer, without limitation, to an arbitrary process of at least one of capturing, recording and storing spectroscopic data by the spectrometer device, e.g. by measuring at least one of a transmission, an absorption, a reflection and an emission of the sample as a function of the wavelength, for one or more different wavelengths.

The spectroscopic measurement, in particular in the infrared spectral range, may be a spot measurement. A spot may comprise an area having an arbitrary geometry. For example, the spot may comprise an area from 1 to 500mm². The image data, in particular an image, acquired by using the image device may cover a bigger area, e.g. from 0.01 m to 10 m, than the spectroscopic measurement. The spectroscopic measurement and acquiring of the image data can occur on different time points T1 and T2. The mixing ratio may be constant at T1 and T2.

The term “spectrometer device” as used herein is a broad term and is to be given its ordinary and customary meaning to a person of ordinary skill in the art and is not to be limited to a special or customized meaning. The term specifically may refer, without limitation, to an apparatus configured for acquiring spectroscopic data of at least one sample within at least one spatial measurement range. The spectrometer device as used in step i. may in particular be a near-infrared spectrometer device. The spectrometer device may specifically be configured for detecting electromagnetic radiation in the near-infrared range. The spectrometer device may be configured for performing at least one spectroscopic measurement on the sample. The spectrometer device may in particular comprise at least one detector device comprising at least one optical element and a plurality of photosensitive elements. The optical element may specifically be configured for separating incident light, specifically electromagnetic radiation in the near-infrared range, into a spectrum of constituent wavelength components. Each photosensitive element may be configured for receiving at least a portion of one of the constituent wavelength components and for generating a respective detector signal depending on an illumination of the respective photosensitive element by the at least one portion of the respective constituent wavelength component. The detector signal, specifically the signal intensity, may together with the corresponding wavelength form part of the spectroscopic data. The spectrometer device may be or may comprise a dispersive spectrometer device that may analyze the radiation of a sample illuminated with a broadband illumination. However, additionally or alternatively, further configurations and/or arrangements of the spectrometer device are feasible. As an example, the sample may be illuminated with light of a limited number of different wavelengths and the spectrometer device may comprise a broadband detector. In particular, the spectrometer device may be a Fourier-Transform spectrometer, specifically a Fourier-Transform infrared spectrometer. Thus, narrow-band light sources may be used, such as at least one light emitting diode (LED) and/or at least one laser, for illuminating the sample. Specifically, the spectrometer device may be configured for determining the spectrum by measuring and processing an interferogram, particularly by applying at least one Fourier transformation to the measured interferogram.

The spectrometer device may in particular be embodied as a portable spectrometer device. Specifically, the spectrometer device may be part of a mobile device such as a notebook computer, a tablet or, specifically, a cell phone such as a smart phone. Additionally or alternatively, the mobile device may be or may comprise a smartwatch and/or a wearable computer, also referred to as wearable, e.g. a body-borne computer. Further mobile devices are feasible. The spectrometer device may be at least one of integrated into the mobile device or attachable thereto.

The term “spatial measurement range” as used herein is a broad term and is to be given its ordinary and customary meaning to a person of ordinary skill in the art and is not to be limited to a special or customized meaning. The term specifically may refer, without limitation, to a spatially limited section, which may be spectroscopically examined by the spectrometer device. As an example, the spatial measurement range may be defined as a solid angle or three-dimensional angular segment in space, wherein samples disposed within the solid angle or angular segment may be analyzed by the spectrometer device. A solid angle or angular segment, as an example, may be defined by geometric and/or optical properties of the spectrometer device. Thus, the spatial measurement range may be the field of view of the spectrometer device in which spectroscopic measurements may be performed. A sample positioned within the spatial measurement range may be accessible to spectroscopic analysis by the spectrometer device. Specifically, the spectrometer device may be configured to acquire spectroscopic data on the basis of incident light from within the spatial measurement range. The spatial measurement range may in particular be a three-dimensional spatial section, e.g. a three-dimensional space, such as a cone-shaped spatial section, whose light content may be received and analyzed by the spectrometer device. The spectroscopic data acquired by the spectrometer device may comprise information relating to at least one sample situated within the spatial measurement range of the spectrometer device. Specifically, for spectroscopically analyzing the sample, the spectrometer device may be positioned in close proximity to the sample, such that the spatial measurement range at least partially comprises the sample, e.g. at a distance in the range from 0 mm to 100 mm from the object, specifically in the range from 0 mm to 15 mm.

The term “sample”, also denoted herein as “object”, as used herein is a broad term and is to be given its ordinary and customary meaning to a person of ordinary skill in the art and is not to be limited to a special or customized meaning. The term specifically may refer, without limitation, to an arbitrary item, such as animate or inanimate item, accessible to being imaged by the imaging device as well as being spectroscopically examined by the spectrometer device. The sample may be a single-component sample or a mixture comprising at least two components. Specifically, the sample may be an inhomogeneous sample, e.g. a sample whose chemical composition may vary within the sample such as in a location-dependent manner. Other samples, however, in particular homogeneous samples with only slight or no variations of their chemical composition, are also feasible. The sample may be any material which shows NIR activity. In this case a chemometric model can be obtained, as described in more detail below. If there is no chemometric model for a certain material available, the method may comprise selecting the model for another material which is most similar to material.

The sample may be a solid sample such as a powder or a solid. However, other samples are possible such as liquid samples.

The sample may specifically be or comprise a food item, such as a fruit or a vegetable. For example, the sample may be or may comprise at least one element selected from the group consisting of: food; feed; a vegetable such as a tomato; a fruit such as an apple, a pear; crop such as wheat, corn; waste e.g. in recycling. For example, the sample may specifically be or comprise a body part, such as the skin.

For example, the method may be used in recycling. The sample may comprise waste such as comprising plastic. The method may comprise obtaining at least one item of identification information on the sample by using image data, e.g. at least one item of identification information relating to the sample shape such as one or more of granularity, color and surface roughness. The chemometric model for analyzing the spectroscopic data may be selected considering the item of identification information.

The term “imaging device” as used herein is a broad term and is to be given its ordinary and customary meaning to a person of ordinary skill in the art and is not to be limited to a special or customized meaning. The term specifically may refer, without limitation, to an arbitrary device configured for recording or capturing image data and/or capturing 2D or 3D spatial information on at least one sample and/or a scene. The imaging device may be or may comprise at least one camera having one or more imaging sensors, specifically one or more CCD or CMOS imaging sensors, for acquiring the image data. The camera may specifically comprise at least one camera chip, such as at least one CCD chip and/or at least one CMOS chip configured for recording images. The camera may comprise a one-dimensional or two-dimensional array of imaging sensors, such as pixels, which may e.g. be arranged on the camera chip. As an example, the camera may comprise at least 100 pixels in at least one dimension, such as at least 100 pixels in each dimension. As an example, the camera may comprise an array of imaging sensors comprising at least 100 imaging sensors in each dimension, specifically at least 300 imaging sensors in each dimension. For example, the camera may be a color camera, comprising color pixels, wherein each color pixel comprises at least three color sub-pixels sensitive for different colors. For example, the camera may comprise black and white pixels and/or color pixels. The color pixels and the black and white pixels may be combined internally in the camera. The camera may be a camera of a mobile device. The invention specifically shall be applicable to cameras as usually used in mobile devices such as notebook computers, tablets or, specifically, cell phones such as smart phones. Specifically, the camera may be part of a mobile device which, besides the at least one camera, comprises one or more data processing devices such as one or more processors. The mobile device, specifically may have at least one function different from the spectroscopic function, such as a mobile communication function, e.g., the function of a cell phone. Other cameras, however, are feasible. As outlined above, the spectrometer device may also be part of a mobile device. In particular, both the camera and the spectrometer device may be part of the mobile device, specifically a smart phone. The camera, besides at least one camera chip or imaging chip, may comprise further elements, such as one or more optical elements, e.g. one or more lenses. As an example, the camera may be a fix-focus camera, having at least one lens, which is fixedly adjusted with respect to the camera. Alternatively, however, the camera may also comprise one or more variable lenses, which may be adjusted, automatically or manually.

Alternatively or in addition, the imaging device may be or may comprise at least one LIDAR-based imaging device, wherein LIDAR stands for Light Detection and Ranging or Light Imaging, Detection and Ranging. The LIDAR-based imaging device may comprise at least on laser source, e.g. at least one tunable laser diode, for illuminating the object or at least one part of the object. The LIDAR-based imaging device may further comprise at least one localization unit configured for determining at least one distance of the illuminated part of the object from the imaging device and/or from at least one further point or location in space. The localization unit may in particular comprise at least one sensor element, e.g. a photo diode, configured for detecting at least one laser beam that was emitted from the laser source and reflected by the object. Determination of the distance, and thus generation of the image data as may comprise processing the light beam reflected by the object and/or at least one reference light beam and/or the corresponding signals detected by the at least one sensor element.

The term “image data” as used herein is a broad term and is to be given its ordinary and customary meaning to a person of ordinary skill in the art and is not to be limited to a special or customized meaning. The term specifically may refer, without limitation, to spatially resolved one-dimensional, two-dimensional or even three-dimensional optical information. The image data may comprise a plurality of electronic readings from the imaging device, such as from the imaging sensors, e.g. the pixels of the camera chip, and/or from the sensor elements of the LIDAR-based imaging device. In particular, the image data may comprise a plurality of numerical values corresponding to the electronic readings from the imaging device. The electronic readings, specifically the numerical values, may relate to at least one optical property of at least one object within a field of view of the imaging device. The image data may comprise at least one array of information values, such as grey scale values and/or color information values. Alternatively or in addition, the information values comprised by the image data may comprise distance values, each indicating a distance between a part of the object and at least one reference point such as the imaging device, in particular the LIDAR-based imaging device.

The term “acquiring image data” as used herein is a broad term and is to be given its ordinary and customary meaning to a person of ordinary skill in the art and is not to be limited to a special or customized meaning. The term specifically may refer, without limitation, to an arbitrary process of capturing or recording image data by the imaging device, specifically the camera, e.g. in the form of electronic readings as generated by the imaging sensors in response to illumination.

The term “field of view” as used herein is a broad term and is to be given its ordinary and customary meaning to a person of ordinary skill in the art and is not to be limited to a special or customized meaning. The term specifically may refer, without limitation, to a spatially limited section, whose content may be imaged by the imaging device. Specifically, the image data generated by the imaging device may comprise spatially resolved optical information relating to the objects located within the field of view of the imaging device. The field of view may in particular be a three-dimensional spatial section that is accessible to the imaging device. Specifically, a scene comprised by the field of view may be imaged by the imaging device.

The term “scene” as used herein is a broad term and is to be given its ordinary and customary meaning to a person of ordinary skill in the art and is not to be limited to a special or customized meaning. The term specifically may refer, without limitation, to an optical content of the field of view of the imaging device. Specifically the scene may comprise one or more objects, such as the sample mentioned with respect to step i. above, wherein the at least one sample in the scene may be imaged by the imaging device. The scene, specifically, may comprise a plurality of objects, having a specific arrangement, wherein the objects and their arrangement may be imaged by the imaging device, thereby generating at least one image. As part of method step ii., image data of a scene within a field of view of the imaging device is acquired, the scene comprising at least a part of the sample and at least a part of the spatial measurement range of the spectrometer device. The sample of step i. may at least partially be visible in the image data of step ii. The field of view of the imaging device and the spatial measurement range of the spectrometer device may, thus, at least partially overlap.

A spatial relationship between the field of view of the imaging device and the spatial measurement range of the spectrometer device may be known and may be used e.g. in step iii., such as offset between the field of view of the imaging device and the spatial measurement range of the spectrometer device and/or at least one angle between the field of view of the imaging device and the spatial measurement range of the spectrometer device. A position and/or an object in the field of view of the imaging device may also be located in the spatial measurement range of the spectrometer device, or vice a versa. Specifically the sample, or at least a part thereof, may be situated in both the field of view of the imaging device and the spatial measurement range of the spectrometer device. The sample, or at least a part thereof, may thus be spectroscopically examined by the spectrometer device as well as at least partially be imaged by the imaging device.

The term “item of identification information” as used herein is a broad term and is to be given its ordinary and customary meaning to a person of ordinary skill in the art and is not to be limited to a special or customized meaning. The term specifically may refer, without limitation, to an arbitrary item of information derived from the image data and/or the spectroscopic data suitable for identifying the sample. The item of identification information may comprise at least one of: at least one item of information about a degree of inhomogeneity, at least one item of information about components of the sample, at least one item of information about a mixture of the components, at least one item of information about a mixing ratio of the components. The item of identification information may be or may comprise at least one item of information on at least one of: a type of the sample, a boundary of the sample within the scene, a size of the sample, an orientation of the sample. A large variety of identification information may be derived from the image data and/or the spectroscopic data.

For example, the item of identification information may comprise information about one or more of a crop species, a crop variety, food or feed type, waste or waste type e.g. in recycling. Other applications, however, are possible.

For example, the method may comprise applying at least one spectroscopic analysis to the spectroscopic data for deriving the at least one item of identification information from the spectroscopic data. As an example, the spectroscopic analysis may comprise analyzing the spectroscopic data to determine at least one peak within the spectroscopic data reflecting a global or local maximum of the transmission, the absorption, the reflection and/or the emission of the sample. The spectroscopic analysis may further comprise identifying the at least one corresponding wavelength. Furthermore, the spectroscopic analysis may comprise determining the chemical composition of the object, e.g. by comparing the identified peaks to at least one predetermined peak or at least one predetermined set of peaks. The spectroscopic analysis of the spectroscopic data may specifically be performed using at least one spectroscopic evaluation algorithm.

For example, the method may comprise applying at least one sample recognition algorithm to the image data for deriving the at least one item of identification information from the image data. The term “image” as used herein is a broad term and is to be given its ordinary and customary meaning to a person of ordinary skill in the art and is not to be limited to a special or customized meaning. The term specifically may refer, without limitation, to an arbitrary representation, e.g. a one-dimensional, two-dimensional or three-dimensional representation, of at least one optically detectable property of the sample. In particular, the image may comprise a graphical representation of the scene within the field of view of the imaging device. The image may specifically be displayed, e.g. on a display device such as a screen of a mobile device, e.g. the mobile device that may comprise the imaging device. The image specifically may comprise the image data mentioned in step ii., or a part thereof, and/or may be derived from the image data or a part thereof. The image may in particular represent at least one visual property of the sample. The image data may comprise information of at least one of:
at least one image derived from the image data of step ii.;
at least one item of spatial information on the spatial measurement range within the scene, specifically an indication of the spatial measurement range at which the spectroscopic data was acquired within an image;
at least one item of information on: a type of the object, a boundary of the object within the scene, a size of the object, an orientation of the object, a color of the object, a texture of the object, a shape of the object, a contrast of the object, a volume of the object, a region of interest of the object;
at least one item of orientation information on the at least one object, specifically an indication of an orientation of the spectrometer device relative to the at least one object;
at least one item of direction information, specifically an indication of a direction between the spectrometer device and the at least one object;
at least one item of resemblance information on the object, specifically resemblance information on at least one shared property, which is shared between different regions of the object.

The expression “analyzing the image data” as used herein is a broad term and is to be given its ordinary and customary meaning to a person of ordinary skill in the art and is not to be limited to a special or customized meaning. The expression specifically may refer, without limitation, to determining at least one item of identification information at least partially on the basis of the image data acquired by the imaging device in step ii. For example, an image analysis of a photo of the sample to be measured may be performed. Such analysis may yield one or more of the kind of sample, its degree of inhomogeneity, in case of mixtures the mixture components and their mixing ratio.

The item of identification information may in particular be derived by using at least one sample recognition algorithm, such as an image recognition algorithm and/or a trained model configured for recognizing or identifying the sample, e.g. by using artificial intelligence, such as an artificial neural network. The sample recognition algorithm may specifically comprise at least one sample recognition algorithm for determining the type of the at least one sample. For example, the sample recognition algorithm may identify the type of the sample, e.g. a category or kind of the object such as the sample being an apple, an orange or another type of fruit or vegetable, a human body part, such as a hand or a face. Further types of sample are possible, in particular further kinds of food samples.

As an example, the image may contain information on the location of the acquisition of the spectroscopic data and/or the result of the evaluation of the spectroscopic data, e.g. composition information derived from the spectroscopic data. The image, thus, may visually indicate the scene, or a part thereof, as well as information derived from the spectroscopic data acquired in step i., optionally with position information regarding the location of acquisition of the information. Thus, the image may contain an overlap between the sample visible in the scene, and one or more locations in which one or more spectroscopic measurements were performed, including, the results of the spectroscopic measurements and/or one or more items of information derived from the spectroscopic measurements.

The item of identification information may be determined using a user selection and/or a user feedback. For example, the user selection and/or the user feedback may be performed by using at least one user interface. The term “user interface” as used herein is a broad term and is to be given its ordinary and customary meaning to a person of ordinary skill in the art and is not to be limited to a special or customized meaning. The term may refer, without limitation, to an element or device which is configured for interacting with its environment, such as for the purpose of uni-directionally or bidirectionally exchanging information, such as for exchange of one or more of data or commands. For example, the user interface may be configured to share information with a user and to receive information by the user. The user interface may be a feature to interact visually with a user, such as a display, or a feature to interact acoustically with the user. The user interface, as an example, may comprise one or more of: a human-machine interface such as a display, a screen, a keyboard, a voice interface, touchpad, or port via which the user can provide the portion of digital information data, a graphical user interface; a data interface, such as a wireless and/or a wire-bound data interface.

The identification information may comprise information on at least one region of interest of the sample. The region of interest may be identified as such e.g. by the image recognition algorithm and/or the trained model. As an example, the region of interest may be or may comprise an irregularity and/or an unexpected feature. Further regions of interest are possible. The region of interest may e.g. be a mole on a stretch of human skin, such as on a hand or leg. The method may provide a step of providing at least one item of guidance information indicating the region of interest to the user, e.g. on the display of the mobile device. The item of guidance information may in particular prompt the user to perform step i. of the method on the region of interest. Using application-specific spectroscopic evaluation algorithms may provide specific information on the region of interest to the user, e.g. medical information and/or medical guidance e.g. cancer diagnostic information on the mole.

The item of identification information may comprise at least one item of resemblance information on the sample, specifically resemblance information on at least one shared property, which is shared between different regions of the sample. In particular, the item of identification information may comprise information on different regions of the sample that share at least one common property. The property may be a quality identified in the image data, particularly in the image. The shared property may e.g. a common color that is shared between different regions of the object while further regions of the object show different colors. The shared property identified in the image data, e.g. similar image information, may imply shared and/or similar spectroscopic data, e.g. similar spectral information. The method may comprise predicting spectroscopic data and/or at least spectroscopically derivable property for regions of the object, which resemble each other in at least one property of the image data. The method may further comprise checking and/or refining the prediction, e.g. by guiding the user to acquire spectroscopic data on the further regions with the shared property. As an example, the sample may be an apple comprising regions of different colors. As part of the method, the regions sharing a red color may be identified as an item of resemblance information. The spectroscopic data acquired for one of the regions may indicate a particular sugar content, e.g. a sugar content that exceeds the sugar content of further regions of different color, e.g. of green color. As part of the method, the sugar content of the further red regions may be predicted. Further, the user may be guided to acquire spectroscopic data on the further red regions to check and/or refine the prediction and/or possible further predictions.

Between the possible repetitions of steps i. and ii., at least one of the scene, the field of view, the spatial measurement range and the object may be modified. Thus, as an example, the scene may vary, and/or at least one of the spectrometer device, the imaging device and a device comprising both the spectrometer device and the imaging device, such as a mobile device, as discussed above, may be moved.

Particularly, the method may generate the at least one image of the scene with at least two items of spectroscopic object information and corresponding spatial information on the spatial measurement range within the image for each item of spectroscopic object information. Further, the image derived from the image data of step ii. may be an image derived from the image data of the repetitions of step ii., specifically at least one of a combined image and a selected image of images derived from the image data of the repetitions of step ii.

As an example, as discussed above, the imaging device and/or the spectrometer device may be moved between, specifically during, the optional repetitions of steps i. and ii. Specifically, in an initial performance of step ii. image data of a first scene may be acquired at a first distance, wherein for the repetitions of step ii. the imaging device and/or the spectrometer device may be moved closer to the sample such that the imaged scenes are subsections of said first scene. In particular, image data corresponding to a wide image may be acquired in the initial performance of step ii. The wide image may comprise the object fully or almost fully. For the further repetitions of step ii. the distance of the imaging device and/or the spectrometer device to the sample may be reduced to at least one second distance, wherein the second distance may allow acquiring spectroscopic data of the object by performing step i. The second distance may be in the range from 0 mm to 100 mm, specifically from 0 mm to 15 mm. The images derived from the image data acquired at the second distance may show subsections of the image derived from the image data acquired in the initial performance of step ii. The method may further comprise tracking a movement of the imaging device, e.g. from the first distance to the at least one second distance, by using the imaging device and a motion tracking software. Specifically, the spatial relation between the image data and/or the spectroscopic data acquired at the at least one second distance with the image acquired at the first distance may be deduced.

As a further example, the imaging device and/or the spectrometer device may be moved across the sample, such as in a fixed distance and/or in a variable distance, e.g. along a scanning path, while performing one or more repetitions of steps i. and ii. By performing step iii., the at least one item of sample information may be obtained, wherein the item of sample information may comprise a plurality of items of chemical information corresponding to a plurality of sites along the scanning path. Again, image data of the sample may be acquired, e.g. in an initial performance of step ii., wherein the scanning path may be comprised by the image derived from the image data. Specifically, the scanning path and/or the spectroscopic object information, specifically the chemical information, may be indicated in the image. This may allow to retrieve the chemical information along the scanning path.

The term “processing device” as generally used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processing device may be configured for processing basic instructions that drive the computer or system. As an example, the processing device may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processing device may be a multi-core processor. Specifically, the processing device may be or may comprise a central processing unit (CPU). Additionally or alternatively, the processing device may be or may comprise a microprocessor, thus specifically the processing device's elements may be contained in one single integrated circuitry (IC) chip. Additionally or alternatively, the processing device may be or may comprise one or more application-specific integrated circuits (ASICs) and/or one or more field-programmable gate arrays (FPGAs) or the like. The processing device specifically may be configured, such as by software programming, for performing one or more evaluation operations.

Step iii., as outlined above, comprises evaluating the spectroscopic data for obtaining the at least one item of sample information by using the processing device. The evaluating comprises processing the spectroscopic data with at least one chemometric model. The chemometric model translates the spectroscopic data into chemical composition information. The chemometric model is selected in accordance with the item of identification information.

Specifically, step iii. may comprise applying at least one spectroscopic evaluation algorithm to the spectroscopic data of step i., wherein the spectroscopic evaluation algorithm is selected in accordance with the item of identification information, specifically in accordance with the type of the at least one object. Based on the item of identification information, the chemometric model may be automatically selected. Thereby, the user experience can be improved. In case of mixtures, the appropriate chemometric models for each component can be selected. The mixing ratio may further be used to calculate any measure for the mixture.

The term “item of sample information” as used herein is a broad term and is to be given its ordinary and customary meaning to a person of ordinary skill in the art and is not to be limited to a special or customized meaning. The term specifically may refer, without limitation, to an arbitrary item of information relating to at least one property of the sample, such as at least one of a chemical, a physical and a biological property, e.g. a material and/or a composition of the sample. The item of sample information may specifically be determined by taking into account the spectroscopic data of the sample as well as the image data of the sample. The item of sample information may specifically relate to a property that may vary within the sample, such that the property may be characteristic for a specific position or spatial range within the sample. The property may, however, show no or only slight variations throughout the sample. The item of sample information may describe the property in a qualitative and/or quantitative manner, e.g. by one or more numerical values. Specifically, the item of sample information may comprise chemical information, in particular a chemical composition, of the sample. The item of sample information may comprise information on the property as well as spatial information on the specific position or spatial range within the sample, where the property was measured. For example, for wheat or corn, the item of sample information may be one or more of: protein content, starch content, fiber content, dry matter and the like. For example, for apples/tomatoes, the item of sample information may be one or more of: dry matter, sugar content, acidity. For example, the method may be used in recycling. The sample may comprise waste such as comprising plastic. The method may comprise obtaining at least one item of identification information on the sample by using image data, e.g. at least one item of identification information relating to the sample shape such as one or more of granularity, color and surface roughness. The chemometric model for analyzing the spectroscopic data may be selected considering the item of identification information.

The term “obtaining at least one item of sample information” as used herein is a broad term and is to be given its ordinary and customary meaning to a person of ordinary skill in the art and is not to be limited to a special or customized meaning. The term specifically may refer, without limitation, to an arbitrary process of determining the at least one item of sample information.

Specifically, for determining the item of sample information the spectroscopic data of step i. and the image data of step ii. may be taken into account.

The terms “evaluating data” and “evaluating information” as used herein in “evaluating spectroscopic data” and “evaluating image information” are broad terms and are to be given their ordinary and customary meaning to a person of ordinary skill in the art and is not to be limited to a special or customized meaning. The terms specifically may refer, without limitation, to an arbitrary process of analyzing the data respectively information, e.g. by applying at least one analysis step, e.g. an analysis step comprising at least one analysis algorithm applied to the data and/or information. Specifically, the data or information may be processed and/or interpreted and/or assessed as part of the analysis step, e.g. by comparing the data or information, or at least a subset thereof, to at least one predetermined value or identifying at least one global or local maximal or minimal value.

The term “chemometric model” as used herein is a broad term and is to be given its ordinary and customary meaning to a person of ordinary skill in the art and is not to be limited to a special or customized meaning. The term specifically may refer, without limitation, to at least one model configured for translating the spectroscopic data into chemical composition information. The chemometric model may comprise mathematical and statistical techniques for extracting relevant information from the spectroscopic data. The chemometric model may comprise using one or more of Multiple Linear Regression (MLR), Principal Component Regression (PCR) and Partial Least Square Regression (PLS), modified PLS such as di-PLS (e.g. as described in Ramin Nikzad-Langerodi, Werner Zellinger, Edwin Lughofer, and Susanne Saminger-Platz Analytical Chemistry 2018 90 (11), 6693-6701 DOI: 10.1021/acs.analchem.8b00498 ), Artificial Neural Networks (ANN), Support Vector Machine, Random Forest, (extreme) Gradient Boost Regression and/or Classification, and the like. The chemometric model may be designed as described in Celio Pasquini, “Near Infrared Spectroscopy: Fundamentals, Practical Aspects and Analytical Applications”, J. Braz. Chem. Soc., Vol. 14, No. 2, 198-219, 2003.

The method may be at least partially computer-implemented, specifically step iii. The computer-implemented steps and/or aspects of the invention, may particularly be performed by using a computer or computer network. As an example, step iii. of the method may be fully or partially computer-implemented. The evaluation of the spectroscopic data is performed using at least one chemometric model. In addition, as outlined above, the analysis of the image data may comprise analyzing the item of image information e.g. using at least one identification algorithm. The evaluated spectroscopic data and the analyzed image data may be combined or connected, e.g. in a predetermined manner and/or according to a predetermined algorithm, for obtaining the at least one item of sample information. The chemometric model may in particular comprise at least one trained model. The term “trained model” as used herein is a broad term and is to be given its ordinary and customary meaning to a person of ordinary skill in the art and is not to be limited to a special or customized meaning. The term specifically may refer, without limitation, to a mathematical model which was trained on at least one training data set using one or more of machine learning, deep learning, neural networks, or other form of artificial intelligence.

The term “chemical composition information” as used herein is a broad term and is to be given its ordinary and customary meaning to a person of ordinary skill in the art and is not to be limited to a special or customized meaning. The term specifically may refer, without limitation, to information about one or more of: components of the mixture, mixing ratio, presence or absence of at least one chemical component and the like.

The method may comprise providing at least one chemometric model. The method may comprise providing a plurality of chemometric models for different items of identification information. The term “providing” as used herein is a broad term and is to be given its ordinary and customary meaning to a person of ordinary skill in the art and is not to be limited to a special or customized meaning. The term specifically may refer, without limitation, to retrieving and/or determining and/or selecting a chemometric model.

The term “retrieving” as used herein is a broad term and is to be given its ordinary and customary meaning to a person of ordinary skill in the art and is not to be limited to a special or customized meaning. The term specifically may refer, without limitation, to the process of a system, specifically a computer system, generating data and/or obtaining data from an arbitrary data source, such as from a data storage, from a network or from a further computer or computer system. The retrieving specifically may take place via at least one computer interface, such as via a port such as a serial or parallel port. The retrieving may comprise several sub-steps, such as the sub-step of obtaining one or more items of primary information and generating secondary information by making use of the primary information, such as by applying one or more algorithms to the primary information, e.g. by using a processor. For example, the providing comprises retrieving the chemometric model from at least one database e.g. from a cloud. The term “database” as used herein is a broad term and is to be given its ordinary and customary meaning to a person of ordinary skill in the art and is not to be limited to a special or customized meaning. The term specifically may refer, without limitation, to an arbitrary collection and/or accumulation of information. Specifically, the database may be or may comprise an accumulation of information that may be sorted and/or linked such as to facilitate a search of information within the database. Therein the term “sorted” may generally refer to a referencing and/or cross-referencing of information. The database may comprise at least one lookup table and/or at least one spreadsheet and/or at least one electronically managed chart. The database may comprise a plurality of chemometric models. The chemometric models may be accessible via the cloud and may be selected in accordance with the item of identification information.

The method may comprise at least one calibration step. The calibration step comprises generating the chemometric model. The calibration step may be performed for each expected single component of the sample.

The chemometric model may be selected automatically. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user.

In case of mixtures, the chemometric model for each component may be selected.

In case of mixtures, the image data may comprises information about a mixing ratio. The mixing ratio may be used for determining the item of sample information for the mixture
by aggregating the chemometric models depending on mixing ratio, wherein the aggregating comprises adding and weighing according to the mixing ratio, and/or
as input parameter for at least one forward model.

The forward model may be designed to use an input parameter and to predict and/or to simulate a spectrum. For example, the forward model may use the mixing ratio as input parameter.

The method may comprise selecting a chemometric model for an item of identification information similar to the determined item of identification information in case no chemometric model for the determined item of identification information is available.

Step iii) of the method may further comprise taking into account information of at least one further sensor in obtaining the at least one item of sample information. The further sensor information may e.g. comprise gyroscopic information and/or GPS information. The further sensor, specifically the gyroscope may be part of the mobile device. Additionally or alternatively, the further sensor information may be provided by the mobile device, e.g. the GPS information. The further sensor information may e.g. be taken into account by checking, verifying or assessing the image data.

The method may further comprise providing at least one output depending on the item of sample information and/or the selected chemometric model by using at least one user interface. The method may further comprise providing the at least one item of sample information on the at least one sample, specifically optically providing the at least one item of sample information on the at least one sample via a display device. Specifically, the item of sample information may be displayed e.g. on a display device such as a screen of a mobile device, e.g. the mobile device that may comprise the imaging device and/or the spectrometer device.

For example, the method may be performed as follows:
In a 1 step, the chemometric model may be generated. For example, in case of determining an acidity in an apple or a sugar content in tomatoes, it may be necessary to perform an individual calibration for crop-parameter combination. Next, the imaging device may take an image of a sample, e.g. via a camera. An image recognition of the sample may be conducted. In a parallel series of steps, the spectrometer device may make an IR spectroscopy measurement of the same sample. The spectroscopic data of the sample may be obtained from the IR spectroscopy measurement. Next, an identification of one or more of crop species, crop variety, or food/feed type may be done only based on the results of the image recognition. A chemometric model may be selected using the information on one or more of the crop species, crop variety, food/feed type. Then this selected chemometric model may be applied on the spectroscopic data. Finally, an item of sample information specific for the (single-component) sample may be obtained, also depending on the selected chemometric model. For example, for wheat or corn, the item of sample information may be one or more of: protein content, starch content, fiber con-tent, dry matter and the like. For example, for apples/tomatoes, the item of sample information may be one or more of: dry matter, sugar content, acidity.

For example, the method may be performed as follows:
In a separate calibration step, the chemometric model may be generated. For example, in case of acidity in apple or sugar content in tomatoes, it may be necessary to perform an individual calibration for crop-parameter combination. Next, the imaging device may take an image of a sample, e.g. via a camera. An image recognition of the sample may be conducted. In a parallel series of steps, the spectrometer device may make an IR spectroscopy measurement of the same sample. The spectroscopic data of the sample may be obtained from the IR spectroscopy measurement. An identification of one or more of crop species, crop variety, or food/feed type may be done based on the result of the image recognition and/or the IR spectrum. No specific order whether first IR spectrum or first image recognition is performed. In some cases (e.g. for wheat or apple), the crop species can be distinguished via IR. In other cases (apple or pear, both have similar IR spectra), the crop species can be distinguished via image recognition. A chemometric model may be selected using the information on one or more of the crop species, crop variety, food/feed type. Then this selected chemometric model may be applied on the spectroscopic data. Finally, an item of sample information specific for the sample may be obtained, also depending on the selected chemometric model.

For example, the method may be performed as follows:
In a separate calibration step, the chemometric model may be generated. For example, in case of acidity in apple or sugar content in tomatoes, it may be necessary to perform an individual calibration for crop-parameter combination. Next, the imaging device may take an image of a sample, e.g. via a camera. An image recognition of the sample may be conducted. In a parallel series of steps, the spectrometer device may make an IR spectroscopy measurement of the same sample. The spectroscopic data of the sample may be obtained from the IR spectroscopy measurement. An identification of one or more of crop species, crop variety, or food or feed type may be done first based on the result of the image recognition and/or IR spectrum, and secondly, additionally, based on user selection or user feedback. Specifically, in case of multiple options, especially in case of crop species/varieties difficult to identify via image recognition and/or IP spectrum (e.g. in case the sample is a white powder and can be wheat flour or plastic powder; e.g. apple vs. pear, both are quite similar), the user may input additional knowledge and can select the best option. E.g. the user may select, e.g. by click in the user interface, on “apple” instead of “pear”. A chemometric model may be selected using the information on one or more of the crop species, crop variety, food or feed type. Then this selected chemometric model may be applied on the spectroscopic data. Finally, an item of sample information specific for the sample may be obtained, also depending on the selected chemometric model.

For example, the method may be performed as follows:
In a separate calibration step, the chemometric model may be generated. For example, in case of acidity in apple or sugar content in tomatoes, it may be necessary to perform an individual calibration for crop-parameter combination. The calibration may be done for the single component. The imaging device may take an image of a sample, e.g. via a camera. Next, an image recognition of the sample may be conducted. In a parallel series of steps, the spectrometer device makes an IR spectroscopy measurement of the same sample. The spectroscopic data of the sample are obtained from the IR spectroscopy measurement. An identification of one or more of crop species, crop variety, or food/feed type may be done only based on the results of the image recognition. At the same time or parallel with the identification of crop species, crop variety, or food/feed type, the mixing ratio may also determined based on the results of the image recognition. A chemometric model may be selected based on one or more of the crop species, crop variety, food/feed type. The chemometric models (data models) may be aggregated depending on mixing ratio information from image recognition: E.g. if the sample has a 30% rye and 70% wheat ratio, the method may comprise aggregating the separate models through adding and weighing according to mixing ratio (e.g. 30% rye chemometric model plus 70% wheat chemometric model). In case of species and/or materials which are different to each other, e.g. plastic and wheat, the method may comprise “back-calculation” for single components within the mixture. Additionally or alternatively, a forward model may be used, e.g. the determined mixing ratio may be used as input parameter. Finally, an item of sample information specific for the (mixture) sample may be obtained, also depending on the selected chemometric model. The item of sample information may comprise an information of the mixing ratio. In case of species and/or materials which are different to each other, e.g. plastic and wheat, the method may comprise “back-mixing of the models” (e.g. for wheat and green wheat with pesticides).

For example, the method may be performed as follows:
In a separate calibration step, the chemometric model may be generated. For example, in case of acidity in apple or sugar content in tomatoes, it may be necessary to perform an individual calibration for crop-parameter combination. The calibration may be done for the single component. The imaging device may take an image of a sample, e.g. via a camera. Next, an image recognition of the sample may be conducted. In a parallel series of steps, the spectrometer device makes an IR spectroscopy measurement of the same sample. The spectroscopic data of the sample are obtained from the IR spectroscopy measurement. An identification of one or more of crop species, crop variety, or food or feed type may be performed using the result of the image recognition and/or IR spectrum. No specific order may be necessary whether first the IR spectrum or first image recognition may be performed. In some cases (e.g. wheat or apple), the crop species can be distinguished via IR. In other cases (e.g. apple or pear, both have similar IR spectra), the crop species may be distinguished via image recognition. The subsequent steps may be performed as described with respect to the previous examples.

For example, the method may be performed as follows:
In a separate calibration step, the chemometric model may be generated. For example, in case of acidity in apple or sugar content in tomatoes, it may be necessary to perform an individual calibration for crop-parameter combination. The calibration may be done for the single component. The imaging device may take an image of a sample, e.g. via a camera. Next, an image recognition of the sample may be conducted. In a parallel series of steps, the spectrometer device makes an IR spectroscopy measurement of the same sample. The spectroscopic data of the sample are obtained from the IR spectroscopy measurement. An identification of one or more of crop species and/or variety identification may be performed using image-supported IR measurements of mixtures. In particular the identification of crop species, crop variety, or food/feed type is done first based on the result of the image recognition and/or IR spectrum, and secondly (additionally) based on user selection or user feedback. In case of multiple options, especially in case of crop species/varieties it may be difficult to identify via image recognition and/or IP spectrum (e.g. if the sample comprises a white powder and can be wheat flour or plastic powder; e.g. apple vs. pear, both are quite similar), the user may input additional knowledge and can select the best option. E.g. the user may select, e.g. by click in the user interface, on “apple” instead of “pear”. The subsequent steps may be performed as described with respect to the previous examples.

As outlined above, the present invention proposes in addition to the spectroscopic measurement using image data. The combining of the spectroscopic measurement and image data may allow for obtaining accurate chemical composition information of the sample, in particular even in case of mixtures, local variations and inhomogeneity of the sample. The method can be used for several uses. The following exemplary examples, are shown for illustration and shall not limit the scope.

For example, in case of silage-Kraftfutter mix, a sample A and a sample B may be taken, e.g. sample A is silage, sample B is Kraftfutter. For example, a farmer wants to know the energy of the feed which is later fed to the cows. The farmer can optimize the silage-Kraftfutter mix. At first, an energy of a component A and an energy of a component B may be measured. So the energy of the final feed mix could be predicted via calculations. However, the energy of the final feed mix usually cannot be exactly determined. The present invention may allow for determining a mixing ratio determined via image recognition plus protein content determined via IR spectroscopy.

For example, in case of wheat-barley or wheat-rye-mixture, different chemometric models may be used. Via image recognition, the ratio wheat-barley or wheat-rye can be determined. E.g. the protein content is different in wheat and in rye. Via image recognition, it may be possible to determine the mixing ratio, and, thus, it may be possible to determine the protein content. The image recognition may allow providing information whether it is a wheat-barley or wheat-rye or another mixture.

For example, in case of food (e.g. on a dish), e.g. Ratatouille or meat-sauce-mixture which can-not be separated, the method may allow measuring the mixture. Thus, it may be possible for determining a e.g. protein content for this dish, not only an average value, but a total value. Specifically, in case of different apple species/varieties (e.g. for apple juice), it may be possible to measure a total value.

In a further aspect of the present invention, a system for obtaining chemical composition information of at least one sample by spectroscopic measurement is disclosed. The system comprises:
I. at least one spectrometer device configured for acquiring spectroscopic data of the sample by using at least one spectrometer device, within at least one spatial measurement range of the spectrometer device;
II. at least one imaging device configured for acquiring image data of a scene within a field of view of the imaging device, the scene comprising at least a part of the sample and at least a part of the spatial measurement range of the spectrometer device;
III. at least one processing device configured for analyzing the image data and/or the spectroscopic data thereby determining at least one item of identification information on the sample, wherein the processing device is configured for evaluating the spectroscopic data for obtaining at least one item of sample information, wherein the evaluating comprises processing the spectroscopic data with at least one chemometric model, wherein the chemometric model is configured for translating the spectroscopic data into chemical composition information, wherein the chemometric model is selected in accordance with the item of identification information.

The system may specifically be used for performing the method of obtaining at least one item of sample information according to the present invention, such as according to any one of the embodiments described above and/or according to any one of the embodiments described further below. Accordingly, regarding terms and definitions, reference may be made to the description of the method of obtaining at least one item of sample information as given above.

The term “system” as used herein is a broad term and is to be given its ordinary and customary meaning to a person of ordinary skill in the art and is not to be limited to a special or customized meaning. The term specifically may refer, without limitation, to a set or an assembly of interacting components, which may interact to fulfill at least one common function. The at least two components may be handled independently or may be coupled or connectable.

The imaging device may comprise at least one camera having one or more imaging sensors, specifically one or more CCD or CMOS imaging sensors, for acquiring the image data of the scene. The imaging device may specifically comprise a one-dimensional or two-dimensional array of imaging sensors, such as pixels, which may e.g. be arranged on the camera chip. Additionally or alternatively, the imaging device may comprise be or may comprise at least one LIDAR-based imaging device. The LIDAR-based imaging device may comprise at least one laser source for illuminating the object and at least one localization unit. The localization unit may comprise at least one sensor element configured for detecting at least one laser beam emitted from the laser source and reflected by the object. The localization unit may be configured for determining at least one distance of the illuminated part of the object from at least one reference point. Determination of the distance, and thus generation of the image data may comprise processing the light beam reflected by the object and/or at least one reference light beam and/or the corresponding signals detected by the at least one sensor element. For further options and/or optional details, reference may be made to the description of the imaging device given above.

The spectrometer device may comprise at least one detector device comprising at least one optical element and a plurality of photosensitive elements, wherein the at least one optical element is configured for separating incident light into a spectrum of constituent wavelength components, wherein each photosensitive element is configured for receiving at least a portion of one of the constituent wavelength components and for generating a respective detector signal de-pending on an illumination of the respective photosensitive element by the at least one portion of the respective constituent wavelength component. Thus, the spectrometer device may analyze incident light after its interaction with the object and generate at least one corresponding detector signal, which may form part of the spectroscopic data. The optical element may comprise at least one wavelength-selective element. The wavelength-selective element may specifically be selected form the group consisting of: a prism; a grating; a linear variable filter; an optical filter, specifically a narrow band pass filter. The detector device may further comprise the plurality of photosensitive elements arranged in a linear array, wherein the array of photosensitive elements comprises a number of 10 to 1000, specifically a number of 100 to 500, specifically a number of 200 to 300, more specifically a number of 256, photosensitive elements. Each photosensitive element may in particular be selected from the group consisting of: a pixelated inorganic camera element, specifically a pixelated inorganic camera chip, more specifically a CCD chip or a CMOS chip; a monochrome camera element, specifically a monochrome camera chip; at least one photoconductor, specifically an inorganic photoconductor, more specifically an inorganic photoconductor comprising Si, PbS, PbSe, Ge, InGaAs, ext. InGaAs, InSb or HgCdTe. Each photosensitive element may be sensitive for electromagnetic radiation in a wavelength range from 760 nm to 1000 μm, specifically in a wavelength range from 760 nm to 15 μm, more specifically in a wavelength range from 1 μm to 5 μm, more specifically in a wavelength range from 1 μm to 3 μm. The spectrometer device may be or may comprise a dispersive spectrometer device that may analyze the radiation of an object illuminated with a broadband illumination, e.g. as described above. However, further configurations and/or arrangements of the spectrometer device are feasible which may in particular affect its components e.g. the detector and/or a source of illumination used. As an example, the object may be illuminated with light of a limited number of different wavelengths. The spectrometer device may comprise a broadband detector. In particular, the spectrometer device may be a Fourier-Transform spectrometer, specifically a Fourier-Transform infrared spectrometer. Thus, narrow-band light sources may be used, such as at least one light emitting diode (LED) and/or at least one laser, for illuminating the object. Specifically, the spectrometer device may be configured for determining the spectrum by measuring and processing an interferogram, particularly by applying at least one Fourier transformation to the measured interferogram.

The spectrometer device and the imaging device may have a known orientation with respect to each other, specifically a fixed orientation. In particular, the spectrometer device and the imaging device may have a known, specifically a fixed spatial relation with respect to each other. Further, the spatial measurement range of the spectrometer device and the field of view of the imaging device may have a fixed spatial relation with respect to each other.

The system may further comprise at least one light source configured for emitting electromagnetic radiation in a wavelength range from 760 nm to 1000 μm, specifically in a wavelength range from 760 nm to 15 μm, more specifically in a wavelength range from 1 μm to 5 μm, more specifically in a wavelength range from 1 μm to 3 μm. The spectrometer device may in particular be referred to as a “near-infrared spectrometer device”.

The processing device of the system is configured for obtaining the at least one item of sample information on the at least one sample. The system may comprise at least one display device configured for providing the at least one item of sample information on the at least one sample. The system may further comprise at least one mobile device, wherein the mobile device comprises the at least one spectrometer device and the at least one imaging device. Thus, the spectrometer device and the imaging device, such as the at least one camera, may both be integrated into the mobile device, such as into a smart phone. The term “mobile device” as used herein is a broad term and is to be given its ordinary and customary meaning to a person of ordinary skill in the art and is not to be limited to a special or customized meaning. The term specifically may refer, without limitation, to a mobile electronics device, more specifically to a mobile communication device such as a cell phone or smart phone. Additionally or alternatively, the mobile device may also refer to a tablet computer or another type of portable computer having at least one camera. The mobile device may particularly have at least one display device, specifically a screen, configured for displaying the item of object information.

The system may further comprise at least one control unit. The term “control unit” as used herein is a broad term and is to be given its ordinary and customary meaning to a person of ordinary skill in the art and is not to be limited to a special or customized meaning. The term specifically may refer, without limitation, to a device or combination of devices capable and/or configured for performing at least one computing operation and/or for controlling at least one function of at least one other device, such as of at least one other component of the system for obtaining at least one item of object information. The control unit may specifically control at least one function of the spectrometer device, e.g. the acquiring of spectroscopic data. The control unit may specifically control at least one function of the imaging device, e.g. the acquiring of image data. The control unit may specifically control the processing device, e.g. the evaluation of the spectroscopic data and/or the image data. Specifically, the at least one control unit may be embodied as at least one processor and/or may comprise at least one processor, wherein the processor may be configured, specifically by software programming, for performing one or more operations. The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming, for controlling and/or performing one or more evaluation operations.

In a further aspect, a computer program is disclosed. The computer program comprises instructions which, when the program is executed by a control unit of the system as disclosed herein, such as according to any one of the embodiments described above and/or according to any one of the embodiments described in further detail below, cause the system to perform the method as disclosed herein, such as according to any one of the embodiments described above and/or according to any one of the embodiments described in further detail below. Thus, as an example, the computer program may cause the system or trigger the system to acquire spectroscopic data by using the spectrometer device in accordance with step i., may cause or trigger the system to acquire, by using the imaging device, image data of the scene according to step ii., and may provide instructions for the system to perform the analysis and/or evaluation, in accordance with step iii. The computer program may in particular comprise instructions, which cause the system to perform step iii. of the method. The computer program may further comprise instructions, which cause the system to perform step i. and step ii. of the method, on its own motion or in response to at least one user action, which may e.g. initiate the acquiring of spectroscopic data in step i. and/or the acquiring of image data in step ii., such as a user interaction like pushing a start button. The computer program may also comprise instructions that cause or trigger the system to prompt the user to provide a specific input. Thus, as an example, the user may be prompted to start the acquisition of the spectroscopic data in step i. and/or may be prompted to start the acquisition of the image data in step ii. Specifically, the computer program may be stored on a computer-readable data carrier and/or on a computer-readable storage medium.

In a further aspect, a computer-readable storage medium is disclosed, comprising instructions which, when the instructions are executed by the control unit of the system as disclosed herein, such as according to any one of the embodiments described above and/or according to any one of the embodiments described in further detail below, cause the control unit to perform the method as disclosed herein, such as according to any one of the embodiments described above and/or according to any one of the embodiments described in further detail below. As used herein, the term “computer-readable storage medium” specifically may refer to a non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Further disclosed and proposed herein is a computer program product having program code means, in order to perform the method according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a trad-able product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically are used only once when introducing the respective feature or element. In most cases, when referring to the respective feature or element, the expressions “at least one” or “one or more” are not repeated, nonwithstanding the fact that the respective feature or element may be present once or more than once.

Further, as used herein, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Summarizing and without excluding further possible embodiments, the following embodiments may be envisaged:
Embodiment 1: A method of obtaining chemical composition information of at least one sample by spectroscopic measurement, the method comprising:
   i. acquiring spectroscopic data of the sample by using at least one spectrometer device, within at least one spatial measurement range of the spectrometer device;
   ii. acquiring, by using at least one imaging device, image data of a scene within a field of view of the imaging device, the scene comprising at least a part of the sample and at least a part of the spatial measurement range of the spectrometer device, and analyzing the image data and/or the spectroscopic data thereby determining at least one item of identification information on the sample by using at least one processing device;
   iii. evaluating the spectroscopic data for obtaining at least one item of sample information by using the processing device, wherein the evaluating comprises processing the spectroscopic data with at least one chemometric model, wherein the chemometric model translates the spectroscopic data into chemical composition information, wherein the chemometric model is selected in accordance with the item of identification information.
Embodiment 2: The method according to the preceding embodiment, wherein the image data comprises at least one of: at least one item of identification information on the at least one sample, at least one item of information about a degree of inhomogeneity, at least one item of information about components of the sample, at least one item of information about a mixture of the components, at least one item of information about a mixing ratio of the components.
Embodiment 3: The method according to any one of the preceding embodiments, wherein the method comprises providing at least one chemometric model.
Embodiment 4: The method according to the preceding embodiment, wherein the providing comprises retrieving the chemometric model from at least one database.
Embodiment 5: The method according to any one of the two preceding embodiments, wherein the method comprises at least one calibration step, wherein the calibration step comprises generating the chemometric model.
Embodiment 6: The method according to the preceding embodiment, wherein the calibration step is performed for each expected single component of the sample.
Embodiment 7: The method according to any one of the preceding embodiments, wherein the method comprises providing a plurality of chemometric models for different items of identification information.
Embodiment 8: The method according to any one of the preceding embodiments, wherein the chemometric model is selected automatically.
Embodiment 9: The method according to any one of the preceding embodiments, wherein, in case of mixtures, the chemometric model for each component is selected.
Embodiment 10: The method according to any one of the preceding embodiments, wherein the method comprises selecting a chemometric model for an item of identification information similar to the determined item of identification information in case no chemometric model for the determined item of identification information is available.
Embodiment 11: The method according to any one of the preceding embodiments, wherein the image data comprises information about a mixing ratio, wherein the mixing ratio is used for determining the item of sample information for the mixture
   by aggregating the chemometric models depending on mixing ratio, wherein the aggregating comprises adding and weighing according to the mixing ratio, and/or
   as input parameter for at least one forward model.
Embodiment 12: The method according to any one of the preceding embodiments, wherein the imaging device is or comprises at least one camera having one or more imaging sensors for acquiring the image data, wherein the imaging device is or comprises at least one CCD imaging sensor or at least one CMOS imaging sensor.
Embodiment 13: The method according to any one of the preceding embodiments, wherein the method comprises applying at least one sample recognition algorithm to the image data for deriving the at least one item of identification information from the image data.
Embodiment 14: The method according to any one of the preceding embodiments, wherein the method comprises applying at least one spectroscopic analysis to the spectroscopic data for deriving the at least one item of identification information from the spectroscopic data.
Embodiment 15: The method according to any one of the preceding embodiments, wherein the item of identification information is determined using a user selection and/or a user feed-back.
Embodiment 16: The method according to any one of the preceding embodiments, wherein the spectrometer device is configured for detecting electromagnetic radiation in the near-infrared range.
Embodiment 17: The method according to any one of the preceding embodiments, wherein the method further comprises providing at least one output depending on the item of sample information and/or the selected chemometric model by using at least one user interface.
Embodiment 18: A system for obtaining chemical composition information of at least one sample by spectroscopic measurement, the system comprising:
   I. at least one spectrometer device configured for acquiring spectroscopic data of the sample by using at least one spectrometer device, within at least one spatial measurement range of the spectrometer device;
   II. at least one imaging device configured for acquiring image data of a scene within a field of view of the imaging device, the scene comprising at least a part of the sample and at least a part of the spatial measurement range of the spectrometer device;
   III. at least one processing device configured for analyzing the image data and/or the spectroscopic data thereby determining at least one item of identification information on the sample, wherein the processing device is configured for evaluating the spectroscopic data for obtaining at least one item of sample information, wherein the evaluating comprises processing the spectroscopic data with at least one chemometric model, wherein the chemometric model is configured for translating the spectroscopic data into chemical composition information, wherein the chemometric model is selected in accordance with the item of identification information.
Embodiment 19: The system according to the preceding embodiment, wherein the system is configured for performing a method according to any one of the preceding embodiments relating to a method.
Embodiment 20: The system according to any one of the preceding embodiments referring to a system, further comprising at least one user interface configured for providing at least one output depending on the item of sample information and/or the selected chemometric model.
Embodiment 21: A computer program comprising instructions which, when the program is executed by a processing device of the system according to any one of the preceding embodiments referring to a system, cause the system to perform the method according to any one of the preceding embodiments referring to a method.
Embodiment 22: A computer-readable storage medium comprising instructions which, when the program is executed by a processing device of the system according to any one of the preceding embodiments referring to a system, cause the system to perform the method according to any one of the preceding embodiments referring to a method.
Embodiment 23: A non-transient computer-readable medium including instructions that, when executed by one or more processors, cause the one or more processors to perform the method according to any one of the preceding embodiments referring to a method.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows a schematic view of a system for obtaining chemical composition information of at least one sample together with a sample;

FIG. 2 shows a flow chart of an embodiment of the method of obtaining chemical composition information of at least one sample;

FIGS. 3A and 3B each illustrate further possible features of the method of obtaining chemical composition information of at least one sample; and

FIGS. 4A to 4D each show a flowchart of an embodiment of the method of obtaining chemical composition information of at least one sample.

### DETAILED DESCRIPTION OF THE EMBODIMENTS

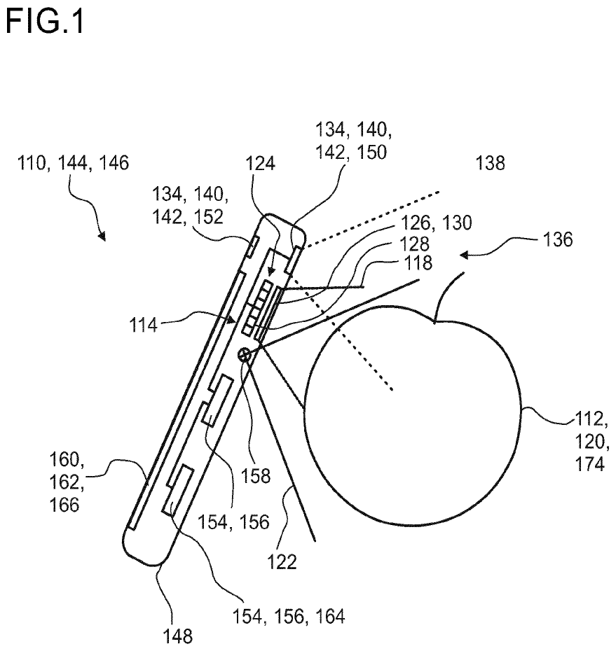

FIG. 1 shows a system 110 for obtaining chemical composition information of at least one sample 112 by spectroscopic measurement. The system 110 comprises at least one spectrometer device 114 configured for acquiring spectroscopic data of the sample 112 by using at least one spectrometer device 114, within at least one spatial measurement range 118 of the spectrometer device 114.

FIG. 1 shows the system 110 with an apple 120 as an exemplary sample 112 placed within the spatial measurement range 118 of the spectrometer device 114 of the system 110. As an example, the spatial measurement range 118 may be defined as a solid angle or three-dimensional angular segment in space, wherein samples 112 disposed within the solid angle or angular segment may be analyzed by the spectrometer device 114. A sample 112 positioned within the spatial measurement range 118 may be accessible to spectroscopic analysis by the spectrometer device 114. Specifically, the spectrometer device 114 may be configured to acquire spectroscopic data on the basis of incident light 122 from within the spatial measurement range 118. The spatial measurement range 118 may in particular be a three-dimensional spatial section, e.g. a three-dimensional space, such as a cone-shaped spatial section, whose light content may be received and analyzed by the spectrometer device 114. The spectroscopic data acquired by the spectrometer device 114 may comprise information relating to the at least one sample 112 situated within the spatial measurement range 118 of the spectrometer device 114. As indicated in FIG. 1, the spectrometer device 114 may be positioned in close proximity to the sample 112 for spectroscopically analyzing the sample 112, such that the spatial measurement range 118 at least partially comprises the sample 112, e.g. at a distance in the range from 0 mm to 100 mm from the sample, specifically in the range from 0 mm to 15 mm.

As illustrated in FIG. 1, the spectrometer device 114 may comprise at least one detector device 124 comprising at least one optical element 126 and a plurality of photosensitive elements 128. The at least one optical element 126 may be configured for separating incident light 122 into a spectrum of constituent wavelength components. Each photosensitive element 128 may be configured for receiving at least a portion of one of the constituent wavelength components and for generating a respective detector signal depending on an illumination of the respective photosensitive element 128 by the at least one portion of the respective constituent wavelength component. Thus, the spectrometer device 114 may analyze incident light 122 after its interaction with the sample 112 and generate at least one corresponding detector signal, which may form part of the spectroscopic data.

As indicated in FIG. 1, the optical element 126 may comprise at least one wavelength-selective element 130. As an example, the wavelength-selective element 130 may specifically be selected form the group consisting of: a prism; a grating; a linear variable filter; an optical filter, specifically a narrow band pass filter. The detector device 124 may further comprise the plurality of photosensitive elements 128 arranged in a linear array, wherein the array of photosensitive elements 128 comprises a number of 10 to 1000, specifically a number of 100 to 500, specifically a number of 200 to 300, more specifically a number of 256, photosensitive elements 128. Each photosensitive element 128 may in particular be selected from the group consisting of: a pixelated inorganic camera element, specifically a pixelated inorganic camera chip, more specifically a CCD chip or a CMOS chip; a monochrome camera element, specifically a monochrome camera chip; at least one photoconductor, specifically an inorganic photoconductor, more specifically an inorganic photoconductor comprising Si, PbS, PbSe, Ge, InGaAs, ext. InGaAs, InSb or HgCdTe. Each photosensitive element 128 may be sensitive for electromagnetic radiation 122 in a wavelength range from 760 nm to 1000 μm, specifically in a wavelength range from 760 nm to 15 μm, more specifically in a wavelength range from 1 μm to 5 μm, more specifically in a wavelength range from 1 μm to 3 μm.

The spectrometer device 114 may be or may comprise a dispersive spectrometer device that may analyze the radiation 122 of a sample 112 illuminated with a broadband illumination, e.g. as described above. However, further configurations and/or arrangements of the spectrometer device 114 are feasible which may in particular affect its components e.g. the detector device 124 and/or a source of illumination used. As an example, the sample 112 may be illuminated with light 122 of a limited number of different wavelengths. The spectrometer device 114 may comprise a broadband detector. In particular, the spectrometer device 114 may be a Fourier-Transform spectrometer, specifically a Fourier-Transform infrared spectrometer. Thus, narrow-band light sources may be used, such as at least one light emitting diode (LED) and/or at least one laser, for illuminating the sample 112. Specifically, the spectrometer device 114 may be configured for determining a spectrum 132 by measuring and processing an interferogram, particularly by applying at least one Fourier transformation to the measured interferogram.

As shown in FIG. 1, the system 110 comprises at least one imaging device 134 configured for acquiring image data of a scene 136 within a field of view 138 of the imaging device 134. The scene 136 comprises at least a part of the sample 112 and at least a part of the spatial measurement range 118 of the spectrometer device 114, as apparent from FIG. 1. The imaging device 134 may be or may comprise at least one camera 140 having one or more imaging sensors 142 for acquiring the image data. The camera 140 may specifically comprise at least one camera chip, such as at least one CCD chip and/or at least one CMOS chip configured for recording images. As an example, the camera 140 may comprise an array of imaging sensors 142 comprising at least 100 imaging sensors 142 in each dimension, specifically at least 300 imaging sensors 142 in each dimension. For example, the camera 140 may be a color camera 140, comprising color pixels, wherein each color pixel comprises at least three color sub-pixels sensitive for different colors. For example, the camera 140 may comprise black and white pixels and/or color pixels. The color pixels and the black and white pixels may be combined internally in the camera 140. The camera 140, besides at least one camera chip or imaging chip, may comprise further elements, such as one or more optical elements, e.g. one or more lenses (not shown). As an example, the camera 140 may be a fix-focus camera 140, having at least one lens, which is fixedly adjusted with respect to the camera 140. Alternatively, however, the camera 140 may also comprise one or more variable lenses, which may be adjusted, automatically or manually.

As depicted in FIG. 1, the imaging device 134 may specifically be the camera 140 of a mobile device 144. As further illustrated in FIG. 1, the spectrometer device 114 may in particular be embodied as a portable spectrometer device 114. Specifically, the spectrometer device 114 may be part of a mobile device 144 such as a notebook computer, a tablet or, specifically, a cell phone such as a smart phone 146. Additionally or alternatively, the mobile device 144 may be or may comprise a smartwatch and/or a wearable computer, also referred to as wearable, e.g. a body-borne computer. Further mobile devices 144 are feasible. As shown in FIG. 1, the spectrometer device 114 may be integrated into the mobile device 144. Additionally or alternatively, the spectrometer device 114 may be attachable thereto. Thus, both the camera 140 and the spectrometer device 114 may be part of the mobile device 144, specifically the smart phone 146. The invention specifically shall be applicable to cameras 140 as usually used in mobile devices 144 such as notebook computers, tablets or, specifically, cell phones such as smart phones 146. The smart phone 146 may further comprise a housing 148, wherein the spectrometer device 114 and the imaging device 134, specifically the camera 140, may be integrally contained within the housing 148. The smart phone 146 may specifically comprise a front camera 150 and a rear camera 152. In particular, the field of view 138 of the front camera 150 may at least partially overlap with the spatial measurement range 118 of the spectrometer device 114 as illustrated in FIG. 1. The mobile device 144 may, besides the at least one camera 140, comprise one or more data processing devices 154 such as one or more processors 156. The mobile device 144 may specifically have at least one function different from the spectroscopic function, such as a mobile communication function, e.g., the function of a cell phone. Other cameras 140, however, are feasible.

In particular, the imaging device 134 may be or may comprise at least one LIDAR-based imaging device 134, wherein LIDAR stands for Light Detection and Ranging or Light Imaging, Detection and Ranging (not shown). The LIDAR-based imaging device 134 may comprise at least on laser source, e.g. at least one tunable laser diode, for illuminating the sample or at least one part of the sample. The LIDAR-based imaging device 134 may further comprise at least one localization unit configured for determining at least one distance of the illuminated part of the sample from the imaging device 134 and/or from at least one further point or location in space. The localization unit may in particular comprise at least one sensor element, e.g. a photo diode, configured for detecting at least one laser beam that was emitted from the laser source and reflected by the sample 112. Determination of the distance, and thus generation of the image data may comprise processing the light beam reflected by the sample 112 and/or at least one reference light beam and/or the corresponding signals detected by the at least one sensor element.

As illustrated in FIG. 1, the spectrometer device 114 and the imaging device 134 may have a known orientation with respect to each other, specifically a fixed orientation. In particular, the spectrometer device 114 and the imaging device 134 may have a known, specifically a fixed spatial relation with respect to each other. Further, the spatial measurement range 118 of the spectrometer device 114 and the field of view 138 of the imaging device 134 may have a fixed spatial relation with respect to each other.

The system 110 may further comprise at least one light source 158 as shown in FIG. 1. The light source 158 may specifically configured for emitting electromagnetic radiation 122 in a wavelength range from 760 nm to 1000 μm, specifically in a wavelength range from 760 nm to 15 μm, more specifically in a wavelength range from 1 μm to 5 μm, more specifically in a wavelength range from 1 μm to 3 μm. The spectrometer device 114 may in particular be referred to as a “near-infrared spectrometer device”. The light source 158 may in particular be configured for illuminating the sample 112, as apparent from FIG. 1. In particular, narrow-band light sources 158 may be used, such as at least one light emitting diode (LED) and/or at least one laser, for illuminating the sample 112.

The system 110 comprises at least one processing device 154 configured for analyzing the image data and/or the spectroscopic data thereby determining at least one item of identification information on the sample 112. The processing device 154 is configured for evaluating the spectroscopic data for obtaining at least one item of sample information, wherein the evaluating comprises processing the spectroscopic data with at least one chemometric model. The chemometric model is configured for translating the spectroscopic data into chemical composition information. The chemometric model is selected in accordance with the item of identification information.

The processing device 154 of the system 110 is configured for obtaining the at least one item of sample information on the at least one sample 112. The system 110 may comprise at least one display device 160 configured for providing the at least one item of sample information on the at least one sample 112. Specifically, e.g. as described above or as described in further detail be-low, the system 110 may comprise at least one mobile device 144, wherein the mobile device 144 comprises the at least one spectrometer device 114 and the at least one imaging device 134. Thus, the spectrometer device 114 and the imaging device 134, such as the at least one camera 140, may both be integrated into the mobile device 144, such as into a smart phone 146. As illustrated in FIG. 1, the mobile device 144 may particularly have the at least one display device 160, specifically a screen 162, configured for displaying the item of object information.

As depicted in FIG. 1, the system 110 may further comprise at least one control unit 164. The control unit 164 may be configured for performing at least one computing operation and/or for controlling at least one function of at least one other component of the system 110 for obtaining chemical composition information of at least one sample 112. The control unit 164 may specifically control at least one function of the spectrometer device 114, e.g. the acquiring of spectroscopic data. The control unit 164 may specifically control at least one function of the imaging device 134, e.g. the acquiring of image data. The control unit 164 may specifically control the processing device 154, e.g. the evaluation of the spectroscopic data and/or the image data.

Specifically, the at least one control unit 164 may be embodied as at least one processor 156 and/or may comprise at least one processor 156, wherein the processor 156 may be configured, specifically by software programming, for performing one or more operations.

The system 110 may further comprise at least one user interface 166. The user interface 166 may e.g. be configured to share information with a user and to receive information by the user. As an example, the user interface 166 may comprise one or more of: a human-machine interface such as a display 160, a screen 162, a keyboard, a voice interface, a touchpad, or port via which the user can provide the portion of digital information data, a graphical user interface; a data interface, such as a wireless and/or a wire-bound data interface. In the embodiment of the system 110 illustrated in FIG. 1, the user interface 166 is a screen 162 of a display device 160. The at least one user interface 166 may specifically be configured for providing at least one output depending on the item of sample information and/or the selected chemometric model.

The system 110 may in particular be configured for performing a method of obtaining chemical composition information of at least one sample 112 by spectroscopic measurement, as described above or as described in more detail further below. The method comprises the following method steps, which specifically may be performed in the given order. However, a different order is also possible. The method may further comprise additional method steps, which are not listed. Further, one or more or even all of the method steps may be performed only once or repeatedly. The method steps are as follows:
i. acquiring spectroscopic data of the sample 112 by using at least one spectrometer device 114, within at least one spatial measurement range 118 of the spectrometer device 114;
ii. acquiring, by using at least one imaging device 134, image data of a scene 136 within a field of view 138 of the imaging device 134, the scene 136 comprising at least a part of the sample 112 and at least a part of the spatial measurement range 118 of the spectrometer device 114, and analyzing the image data and/or the spectroscopic data thereby determining at least one item of identification information on the sample 112 by using at least one processing device 154; and
iii. evaluating the spectroscopic data for obtaining at least one item of sample information by using the processing device 154, wherein the evaluating comprises processing the spectroscopic data with at least one chemometric model, wherein the chemometric model translates the spectroscopic data into chemical composition information, wherein the chemometric model is selected in accordance with the item of identification information.

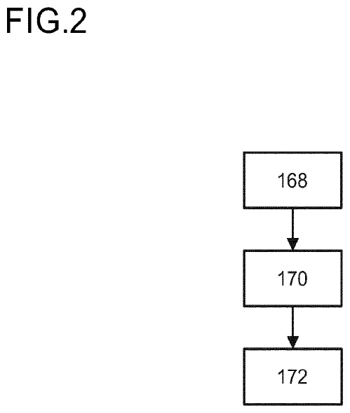

FIG. 2 illustrates the method of obtaining chemical composition information of at least one sample 112 by spectroscopic measurement. In particular, step i. is represented by reference sign 168; step ii. is represented by reference sign 170; and step ii. is represented by reference sign 172.

In addition to the spectroscopic measurement, the method proposes using image data. The combining of the spectroscopic measurement and image data may allow for obtaining accurate chemical composition information of the sample 112, in particular even in case of mixtures, local variations and inhomogeneity of the sample 112. The sample 112 shown within the spatial measurement range 118 of the spectrometer device 114 in FIG. 1 is an apple 120. However, a wide variety of samples 112 is feasible. The sample 112 may be a single-component sample 112 or a mixture comprising at least two components. Specifically, the sample 112 may be an inhomogeneous sample 112, e.g. a sample 112 whose chemical composition may vary within the sample 112 such as in a location-dependent manner. Other samples 112, however, in particular homogeneous samples 112 with only slight or no variations of their chemical composition are also feasible. The sample 112 may be any material, which shows NIR activity. In this case, a chemometric model can be obtained as described in more detail above and/or as described in more detail below. If there is no chemometric model for a certain material available, the model for another material, which is most similar to the material, may be used.

The sample 112 may be a solid sample 112 such as a powder or a solid. However, other samples 112 are possible such as liquid samples 112. The sample 112 may specifically be or comprise a food item, such as a fruit 174 or a vegetable. For example, the sample 112 may be or may comprise at least one element selected from the list consisting of: food; feed; a vegetable such as a tomato; a fruit 174 such as an apple 120, a pear; crop such as wheat, corn; waste e.g. in recycling. For example, the sample 112 may specifically be or comprise a body part 176, such as the skin 178. For example, the method may be used in recycling. The sample may comprise waste such as comprising plastic. The method may comprise obtaining at least one item of identification information on the sample by using image data, e.g. at least one item of identification information relating to the sample shape such as one or more of granularity, color and surface roughness. The chemometric model for analyzing the spectroscopic data may be selected considering the item of identification information.

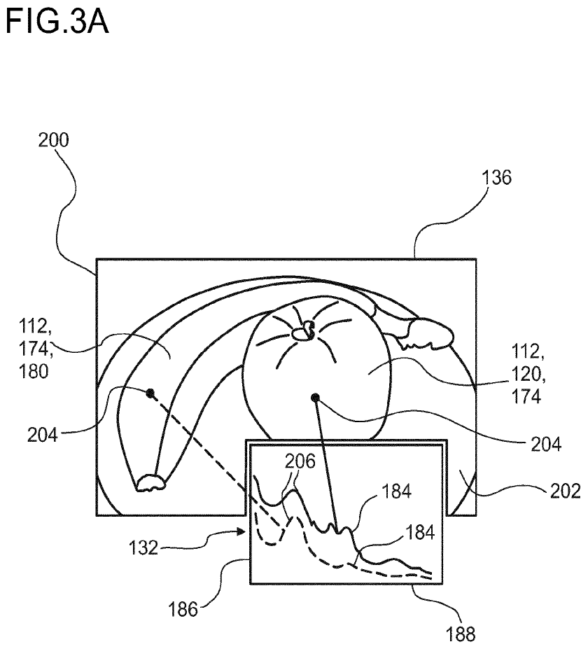

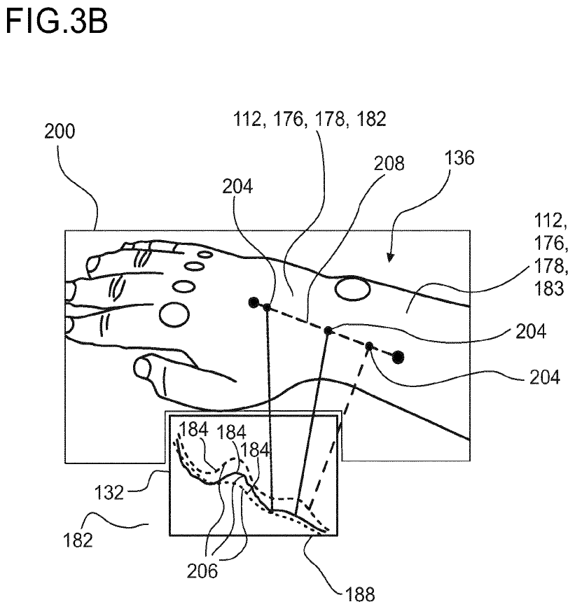

FIGS. 3A and 3B show three possible samples 112, specifically an apple 120, a banana 180 and the skin 178 of a human hand 182 and arm 183, respectively. FIGS. 3A and 3B further illustrate spectroscopic data in the form of spectra 132 acquired with the spectrometer device 114 as part of the spectroscopic measurement of step i. of the method. As part of the spectroscopic measurement, the sample 112 may be illuminated with electromagnetic radiation 122 in the infrared spectral range, specifically in the near infrared spectral range. In particular, the electromagnetic radiation may be in a wavelength range from 760 nm to 1000 μm, specifically in a wavelength range from 760 nm to 15 μm, more specifically in a wavelength range from 1 μm to 5 μm, more specifically in a wavelength range from 1 μm to 3 μm. The spectroscopic measurement may further comprise receiving incident light after interaction with the sample and generating at least one corresponding signal, which may form part of the spectroscopic data. The spectroscopic data may comprise information on at least one optical property or optically measurable property of the sample 112, which is determined as a function of the wavelength, for one or more different wavelengths. More specifically, the spectroscopic data may relate to at least one property characterizing at least one of a transmission, an absorption, a reflection and an emission of the sample. The spectroscopic data may specifically take the form of a signal intensity determined as a function of the wavelength of the spectrum or a partition thereof, such as a wavelength interval, wherein the signal intensity may preferably be provided as an electrical signal, which may be used for further evaluation. The spectroscopic data may, e.g. be graphically represented in the form of a spectral curve 184, wherein the signal intensity I plotted on the y-axis 186 is shown as a function of wavelength λ plotted on the x-axis 188, as depicted in FIGS. 3A and 3B. Specifically, the signal intensity I may correspond to an intensity of reflected electromagnetic radiation 122, e.g. of electromagnetic radiation 122 in the infrared spectral range, with which the sample 112 may be illuminated. The spectral curve 184 may show the reflected intensity I as a function of the wavelength λ, as illustrated in FIGS. 3AB and 3B.

As part of step ii. of the method, image data of a scene within a field of view of the imaging device is acquired. The image data may comprise a plurality of electronic readings from the imaging device, such as from the imaging sensors, e.g. the pixels of the camera chip, and/or from the sensor elements of the LIDAR-based imaging device. In particular, the image data may comprise a plurality of numerical values corresponding to the electronic readings from the imaging device. FIGS. 3A and 3B illustrate image data in the form of a graphical representation, specifically in the form of an image 200, of the scene 136 within the field of view 138 of the imaging device, specifically the camera 140. As apparent from FIG. 3A, the scene 136 may comprise a plurality of objects, having a specific arrangement, wherein the objects and their arrangement may be imaged by the imaging device 134, thereby generating the at least one image 200. Specifically, the image 200 illustrated in FIG. 3A shows a scene 136 comprising an apple 120 and a banana 180 arranged on a plate 202. The image 200 illustrated in FIG. 3B shows a scene 136 comprising part of a human hand 182 and arm 183. The sample of step i. may at least partially be visible in the image data of step ii. The field of view of the imaging device and the spatial measurement range of the spectrometer device may, thus, at least partially overlap.

As shown in FIGS. 3A and 3B, the sample, or at least a part thereof, may be situated in both the field of view 138 of the imaging device 134 and the spatial measurement range 118 of the spectrometer device 114. The sample, or at least a part thereof, may thus be spectroscopically examined by the spectrometer device 114 as well as at least partially be imaged by the imaging device 134. As indicated in FIGS. 3A and 3B, the spectroscopic measurement may be a spot measurement, e.g. of spots 204 having diameters of 8 mm to 2 cm. A spot may comprise an area having an arbitrary geometry. For example, the spot may comprise an area from 1 to 500 mm². The image data, in particular an image 200, acquired by using the image device 134 may cover a bigger area, e.g. from 0.01 m to 10 m, than the spectroscopic measurement. The spectroscopic measurement and acquiring of the image data can occur on different time points T1 and T2. A mixing ratio of the components of the sample 112 may be constant at T1 and T2. As depicted in FIGS. 3A and 3B, the image 200 may contain information on the location, in particular the spot 204, of the acquisition of the spectroscopic data and/or the result of the evaluation of the spectroscopic data, e.g. composition information derived from the spectroscopic data.

The image 200, thus, may visually indicate the scene 136, or a part thereof, as well as information derived from the spectroscopic data acquired in step i., optionally with position information regarding the location of acquisition of the information.

Step ii. of the method comprises analyzing the image data and/or the spectroscopic data thereby determining at least one item of identification information on the sample 112 by using the at least one processing device 154. A large variety of identification information may be de-rived from the image data and/or the spectroscopic data. The item of identification information may comprise at least one of: at least one item of information about a degree of inhomogeneity, at least one item of information about components of the sample 112, at least one item of information about a mixture of the components, at least one item of information about a mixing ratio of the components. The item of identification information may be or may comprise at least one item of information on at least one of: a type of the sample 112, a boundary of the sample 112 within the scene 136, a size of the sample 112, an orientation of the sample 112.

Specifically, at least one spectroscopic evaluation algorithm may be applied to the spectroscopic data as part of a spectroscopic analysis for deriving the at least one item of identification information. The spectroscopic analysis may comprise determining the chemical composition of the sample, e.g. by comparing one or more identified peaks 206 of the spectroscopic data as marked in FIGS. 3A and 3B, to at least one predetermined peak 206 or at least one predetermined set of peaks 206. Additionally or alternatively, the method may comprise applying at least one sample recognition algorithm to the image data for deriving the at least one item of identification information from the image data. The item of identification information may in particular be derived by using at least one sample recognition algorithm, such as an image recognition algorithm and/or a trained model configured for recognizing or identifying the sample 112, e.g. by using artificial intelligence, such as an artificial neural network. For example, the sample recognition algorithm may identify the type of the sample 112, e.g. a category or kind of the object such as the sample 112 being an apple 120, an orange or another type of fruit 174 or vegetable, a human body part 176, such as a hand 182 or a face. Further types of sample 112 are possible, in particular further kinds of food samples 112. As an example, the item of identification information may be determined using a user selection and/or a user feedback, e.g. by using the at least one user interface 166.

As a further example, at least one of the scene 136, the field of view 138, the sample 112 and the spatial measurement range 118 may be modified between the possible repetitions of steps i. and ii. In particular, the scene 136 may vary, and/or at least one of the spectrometer device 114, the imaging device 134 and a device comprising both the spectrometer device 114 and the imaging device 134, such as the mobile device 144 may be moved. Thus, as illustrated in FIGS. 3A and 3B, the method may generate the at least one image 200 of the scene 136 with at least two items of spectroscopic object information and corresponding spatial information on the spatial measurement range 118 within the image 200 for each item of spectroscopic object information. Further, the image 200 derived from the image data of step ii. may be an image 200 derived from the image data of the repetitions of step ii., specifically at least one of a combined image 200 and a selected image 200 of images 200 derived from the image data of the repetitions of step ii. As indicated in FIG. 3B, the imaging device and/or the spectrometer device may in particular be moved across the sample 112 along a scanning path 208, while performing one or more repetitions of steps i. and ii. By performing step iii., the at least one item of sample information may be obtained, wherein the item of sample information may comprise a plurality of items of chemical information corresponding to a plurality of sites along the scanning path 208. Again, image data of the sample 112 may be acquired, e.g. in an initial performance of step ii., wherein the scanning path 208 may be comprised by the image 200 derived from the image data. Specifically, the scanning path 208 and/or the spectroscopic object information, specifically the chemical information, may be indicated in the image 200. This may allow to retrieve the chemical information along the scanning path 208.

Step iii. comprises evaluating the spectroscopic data for obtaining the at least one item of sample information by using the processing device 154. The evaluating comprises processing the spectroscopic data with at least one chemometric model. The chemometric model translates the spectroscopic data into chemical composition information. The chemometric model is selected in accordance with the item of identification information. Step iii. may comprise applying at least one spectroscopic evaluation algorithm to the spectroscopic data of step i., wherein the spectroscopic evaluation algorithm is selected in accordance with the item of identification information, specifically in accordance with the type of the at least one object 112.

The item of sample information may specifically be determined by taking into account the spectroscopic data of the sample as well as the image data of the sample. The item of sample information may specifically relate to a property that may vary within the sample 112, such that the property may be characteristic for a specific position or spatial range within the sample 112. The property may, however, show no or only slight variations throughout the sample 112. The item of sample information may describe the property in a qualitative and/or quantitative manner, e.g. by one or more numerical values. Specifically, the item of sample information may comprise chemical information, in particular a chemical composition, of the sample 112. The item of sample information may comprise information on the property as well as spatial information on the specific position or spatial range within the sample 112, where the property was measured. As an example, for the apple 120 shown in FIG. 3A, the item of sample information may be one or more of: dry matter, sugar content, acidity. As a further example, the item of sample information for wheat or corn may be one or more of: protein content, starch content, fiber content, dry matter and the like. As a further example, the item of sample information for waste, e.g. in recycling, may be one or more of granularity, color and surface roughness.

The chemometric model translates the spectroscopic data into chemical composition information, which may relate to information about one or more of: components of the mixture, mixing ratio, presence or absence of at least one chemical component, and the like. The method may comprise providing the at least one chemometric model, specifically a plurality of chemometric models for different items of identification information. Specifically, the chemometric model may be retrieved from at least one database, e.g. from a cloud. The chemometric models may be accessible via the cloud and may be selected in accordance with the item of identification information. The chemometric model may be selected automatically. As indicated above, the sample 112 may be a single-component sample 112 or a mixture comprising at least two components. In case of mixtures, the chemometric model for each component may be selected. In case of mixtures, the image data may comprises information about a mixing ratio. The mixing ratio may be used for determining the item of sample information for the mixture
by aggregating the chemometric models depending on mixing ratio, wherein the aggregating comprises adding and weighing according to the mixing ratio, and/or
as input parameter for at least one forward model.
   The forward model may be designed to use an input parameter and to predict and/or to simulate a spectrum. For example, the forward model may use the mixing ratio as input parameter.

The method may comprise selecting a chemometric model for an item of identification information similar to the determined item of identification information in case no chemometric model for the determined item of identification information is available. The method may be at least partially computer-implemented, specifically step iii. The chemometric model may comprise mathematical and statistical techniques for extracting relevant information from the spectroscopic data. The chemometric model may in particular comprise at least one trained model. In addition, as outlined above, the analysis of the image data may comprise analyzing the item of image information e.g. using at least one identification algorithm.

The method may comprise at least one calibration step. The calibration step comprises generating the chemometric model. The calibration step may be performed for each expected single component of the sample 112.

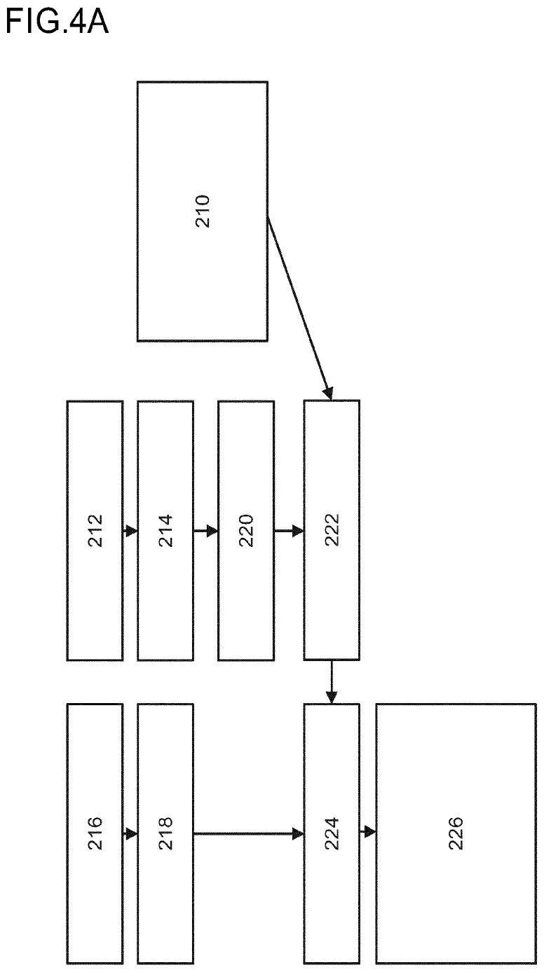

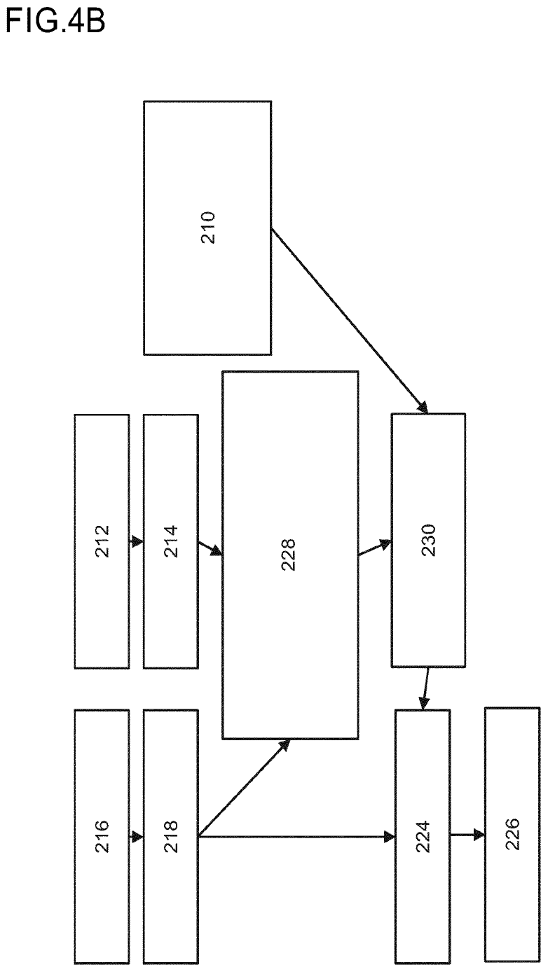

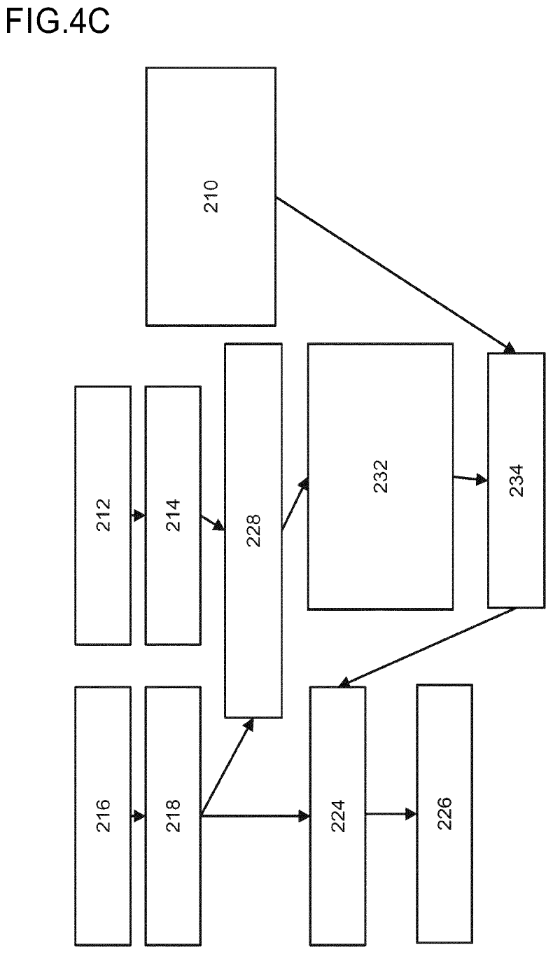

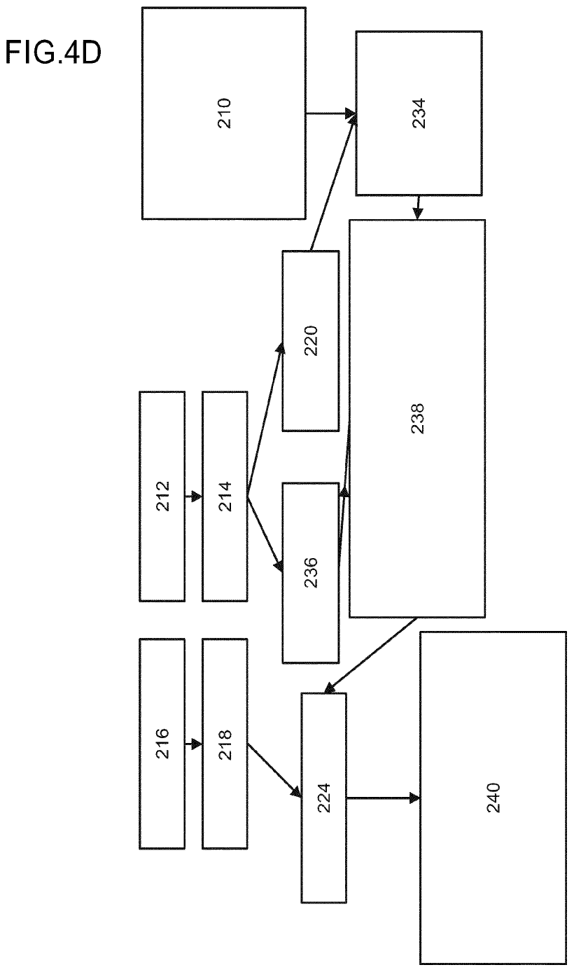

In the following, four embodiments of the method as illustrated in the flowcharts shown in FIGS. 4A to 4D will be described in an exemplary fashion.

As illustrated in FIG. 4A, the method may e.g. be performed as follows:
In a calibration step, the chemometric model may be generated. For example, in case of determining an acidity in an apple 120 or a sugar content in tomatoes, it may be necessary to perform an individual calibration for crop-parameter combination. The calibration step is indicated by reference sign 210. Next, the imaging device 134 may take an image 200 of a sample 112, e.g. via a camera 140. This step is indicated by reference sign 212. An image recognition of the sample 112 may be conducted, as indicated by reference sign 214. In a parallel series of steps, the spectrometer device 114 may make an IR spectroscopy measurement of the same sample 112, as indicated by reference sign 216. The spectroscopic data of the sample may be obtained from the IR spectroscopy measurement, as indicated by reference sign 218. Next, an identification of one or more of crop species, crop variety, or food/feed type may be done only based on the results of the image recognition. This step is indicated by reference sign 220. A chemometric model may be selected using the information on one or more of the crop species, crop variety, food/feed type, as indicated by reference sign 222. Then this selected chemometric model may be applied on the spectroscopic data, as indicated by reference sign 224. Finally, an item of sample information specific for the (single-component) sample may be obtained, also de-pending on the selected chemometric model. For example, for wheat or corn, the item of sample information may be one or more of: protein content, starch content, fiber con-tent, dry matter and the like. For example, for apples/tomatoes, the item of sample information may be one or more of: dry matter, sugar content, acidity. The step of obtaining the item of sample information is indicated by reference sign 226.

As illustrated in FIG. 4B, the method may e.g. be performed as follows:
In a separate calibration step, the chemometric model may be generated. For example, in case of acidity in apple 120 or sugar content in tomatoes, it may be necessary to perform an individual calibration for crop-parameter combination. The calibration step is indicated by reference sign 210. Next, the imaging device 134 may take an image 200 of a sample 212, e.g. via a camera 140. This step is indicated by reference sign 212. An image recognition of the sample 112 may be conducted, as indicated by reference sign 214. In a parallel series of steps, the spectrometer device 114 may make an IR spectroscopy measurement of the same sample 112, as indicated by reference sign 216. The spectroscopic data of the sample 112 may be obtained from the IR spectroscopy measurement, as indicated by reference sign 218. An identification of one or more of crop species, crop variety, or food/feed type may be done based on the result of the image recognition and/or the IR spectrum. No specific order whether first IR spectrum or first image recognition is performed. In some cases (e.g. for wheat or apple 120), the crop species can be distinguished via IR. In other cases (apple 120 or pear, both have similar IR spectra), the crop species can be distinguished via image recognition. The step of identification based on the result of the image recognition and/or the IR spectrum is indicated by reference sign 228. A chemometric model may be selected using the information on one or more of the crop species, crop variety, food/feed type, as indicated by reference sign 230. Then this selected chemometric model may be applied on the spectroscopic data, as indicated by reference sign 224. Finally, an item of sample information specific for the sample 112 may be obtained, also depending on the selected chemometric model. The step of obtaining the item of sample information is indicated by reference sign 226.

As illustrated in FIG. 4C, the method may e.g. be performed as follows:
In a separate calibration step, the chemometric model may be generated. For example, in case of acidity in apple 120 or sugar content in tomatoes, it may be necessary to perform an individual calibration for crop-parameter combination. The calibration step is indicated by reference sign 210. Next, the imaging device 134 may take an image 200 of a sample 112, e.g. via a camera 140. This step is indicated by reference sign 212. An image recognition of the sample 112 may be conducted, as indicated by reference sign 214. In a parallel series of steps, the spectrometer device 112 may make an IR spectroscopy measurement of the same sample 112, as indicated by reference sign 216. The spectroscopic data of the sample 112 may be obtained from the IR spectroscopy measurement, as indicated by reference sign 218. An identification of one or more of crop species, crop variety, or food or feed type may be done first based on the result of the image recognition and/or IR spectrum, as indicated by reference sign 228, and secondly, additionally, based on user selection or user feedback, as indicated by references sign 232. Specifically, in case of multiple options, especially in case of crop species/varieties difficult to identify via image recognition and/or IP spectrum (e.g. in case the sample 112 is a white powder and can be wheat flour or plastic powder; e.g. apple 120 vs. pear, both are quite similar), the user may input additional knowledge and can select the best option. E.g. the user may select, e.g. by click in the user interface 166, on “apple” 120 instead of “pear”. A chemometric model may be selected using the information on one or more of the crop species, crop variety, food or feed type as indicated by reference sign 234. Then this selected chemometric model may be applied on the spectroscopic data, as indicated by reference sign 224. Finally, an item of sample information specific for the sample 112 may be obtained, also depending on the selected chemometric model. The step of obtaining the item of sample information is indicated by reference sign 226.

As illustrated in FIG. 4D, the method may e.g. be performed as follows:
In a separate calibration step, the chemometric model may be generated. For example, in case of acidity in apple or sugar content in tomatoes, it may be necessary to perform an individual calibration for crop-parameter combination. The calibration may be done for the single component. The calibration step is indicated by reference sign 210. The imaging device 134 may take an image 200 of a sample 212, e.g. via a camera 140. This step is indicated by reference sign 212. Next, an image recognition of the sample 112 may be conducted, as indicated by reference sign 214. In a parallel series of steps, the spectrometer device 114 makes an IR spectroscopy measurement of the same sample 112, as indicated by reference sign 216. The spectroscopic data of the sample are obtained from the IR spectroscopy measurement, as indicated by reference sign 218. An identification of one or more of crop species, crop variety, or food/feed type may be done only based on the results of the image recognition. This step is indicated by reference sign 220. At the same time or parallel with the identification of crop species, crop variety, or food/feed type, the mixing ratio may also determined based on the results of the image recognition. This step is indicated by reference sign 236. One or more chemometric models may be selected based on one or more of the crop species, crop variety, food/feed type, as indicated in step 234. The chemometric models (data models) may be aggregated depending on mixing ratio information from image recognition: E.g. if the sample has a 30% rye and 70% wheat ratio, the method may comprise aggregating the separate models through adding and weighing according to mixing ratio (e.g. 30% rye chemometric model plus 70% wheat chemometric model). In case of species and/or materials which are different to each other, e.g. plastic and wheat, the method may comprise “back-calculation” for single components within the mixture. Additionally or alternatively, a forward model may be used, e.g. the determined mixing ratio may be used as input parameter. The step of selecting a chemometric model either based on the aggregated chemometric models or on the forward model is indicated by reference sign 238. Finally, an item of sample information specific for the (mixture) sample 112 may be obtained, also depending on the selected chemometric model. The item of sample information may comprise an information of the mixing ratio. In case of species and/or materials which are different to each other, e.g. plastic and wheat, the method may comprise “back-mixing of the models” (e.g. for wheat and green wheat with pesticides). The step of obtaining an item of sample information specific for the (mixture) sample 112 is indicated by reference sign 240

### LIST OF REFERENCE NUMBERS

110 system
112 sample
114 spectrometer device
118 spatial measurement range
120 apple
122 light
124 detector device
126 optical element
128 photosensitive element
130 wavelength-selective element
132 spectrum
134 imaging device
136 scene
138 field of view
140 camera
142 imaging sensor
144 mobile device
146 smart phone
148 housing
150 front camera
152 rear camera
154 processing device
156 processor
158 light source
160 display device
162 screen
164 control unit
166 user interface
168 step i.
170 step ii.
172 step iii.
174 fruit
176 body part
178 skin
180 banana
182 hand
183 arm
184 spectral curve
186 y-axis
188 x-axis
200 image
202 plate
204 spot
206 peak
208 scanning path
210 “Generation of chemometric model in a separate calibration step (e.g. acidity in apple or sugar content in tomatoes, i.e. you need an individual calibration for crop-parameter combination)”
212 “Take an image of the sample (e.g. via camera)”
214 “Image recognition of the sample”
216 “Make IR spectroscopy measurements of the sample”
218 “Obtaining spectral data from IR”
220 “Crop species/variety or type of food/feed identified based on image recognition”
222 “Selection of chemometric model (data model) depending on result of image recognition”
224 “Application of chemometric model (data model) on the spectral data”
226 “Result for single component: E.g. for wheat/corn: protein content, starch con-tent, fiber content, dry matter etc.; E.g. for apples/tomatoes: dry matter, sugar content, acidity”
228 “Result of image recognition and/or IR spectrum: crop species/variety (e.g. whether it's wheat or rye) or type of food/feed; Details: No order whether first IR spectrum or first image recognition; In some cases (wheat or apple), it can be distinguished via IR; In other cases (apple or pear, both have similar IR spectra), it can only be distinguished via image recognition.”
230 “Selection of chemometric model (data model) depending on result of image recognition and/or IR spectrum”
232 “User selection/user feedback: In case of multiple options (e.g. it's a white powder and can be wheat flour or plastic powder; e.g. apple vs. pear, both are quite similar), but the user knows what it is and can select the best option; relevant in B2C area.”
234 “Selection of chemometric model (data model) depending on result of image recognition and/or IR spectrum and/or user selection/user feedback”
236 “2nd result of image recognition: mixture ratio”
238 “Subvariant A: Aggregating the >1 chemometric models (data models) de-pending on mixture ratio information from image recognition; Subvariant B: (most preferably) forward model, here, the mixture ratio is also required as input”
240 “a) Result for mixture (e.g. the wheat/barley mixture had the protein content X; mixing ratio) and/or b) Only in case of species/materials which are different to each other (e.g. plastic and wheat): result for single components within the mixture based on “back-calculation” in case of subvariant B or “back-mixing of the models” in case of subvariant A”

1. A method of obtaining chemical composition information of at least one sample by spectroscopic measurement, the method comprising:
i. acquiring spectroscopic data of the sample by using at least one spectrometer device, within at least one spatial measurement range of the spectrometer device;
ii. acquiring, by using at least one imaging device, image data of a scene within a field of view of the imaging device, the scene comprising at least a part of the sample and at least a part of the spatial measurement range of the spectrometer device, and analyzing the image data and/or the spectroscopic data thereby determining at least one item of identification information on the sample by using at least one processing device, wherein the image data comprises information about a mixing ratio; and
iii. evaluating the spectroscopic data for obtaining at least one item of sample information by using the processing device, wherein the evaluating comprises processing the spectroscopic data with at least one chemometric model, wherein the chemometric model translates the spectroscopic data into chemical composition information, wherein the chemometric model is selected in accordance with the item of identification information, wherein, in case of mixtures, the chemometric model for each component is selected, wherein the mixing ratio is used for determining the item of sample information for the mixture
by aggregating the chemometric models depending on the mixing ratio, wherein the aggregating comprises adding and weighing according to the mixing ratio, and/or
as input parameter for at least one forward model.

2. The method according to claim 1, wherein the image data comprises at least one of: at least one item of identification information on the at least one sample, at least one item of information about a degree of inhomogeneity, at least one item of information about components of the sample, at least one item of information about a mixture of the components, and at least one item of information about a mixing ratio of the components.

3. The method according to claim 1, wherein the method comprises providing at least one chemometric model.

4. The method according to claim 3, wherein the method comprises at least one calibration step, wherein the calibration step comprises generating the chemometric model.

5. The method according to claim 1, wherein the method comprises providing a plurality of chemometric models for different items of identification information.

6. The method according to claim 1, wherein the method comprises selecting a chemometric model for an item of identification information similar to a determined item of identification information in case no chemometric model for the determined item of identification information is available.

7. The method according to claim 1, wherein the item of identification information is determined using a user selection and/or a user feedback.

8. The method according to claim 1, wherein the method further comprises providing at least one output depending on the item of sample information and/or the selected chemometric model by using at least one user interface.

9. A system for obtaining chemical composition information of at least one sample by spectroscopic measurement, the system comprising:
I. at least one spectrometer device configured for acquiring spectroscopic data of the sample by using at least one spectrometer device, within at least one spatial measurement range of the spectrometer device;
II. at least one imaging device configured for acquiring image data of a scene within a field of view of the imaging device, the scene comprising at least a part of the sample and at least a part of the spatial measurement range of the spectrometer device; and
III. at least one processing device configured for analyzing the image data and/or the spectroscopic data thereby determining at least one item of identification information on the sample, wherein the processing device is configured for evaluating the spectroscopic data for obtaining at least one item of sample information, wherein the evaluating comprises processing the spectroscopic data with at least one chemometric model, wherein the chemometric model is configured for translating the spectroscopic data into chemical composition information, wherein the chemometric model is selected in accordance with the item of identification information.

10. The system according to claim 9, wherein the system is configured for performing a method comprising:
i. acquiring spectroscopic data of the sample by using the at least one spectrometer device, within at least one spatial measurement range of the spectrometer device;
ii. acquiring, by using the at least one imaging device, image data of a scene within a field of view of the imaging device, the scene comprising at least a part of the sample and at least a part of the spatial measurement range of the spectrometer device, and analyzing the image data and/or the spectroscopic data thereby determining at least one item of identification information on the sample by using at least one processing device, wherein the image data comprises information about a mixing ratio; and
iii. evaluating the spectroscopic data for obtaining at least one item of sample information by using the processing device, wherein the evaluating comprises processing the spectroscopic data with at least one chemometric model, wherein the chemometric model translates the spectroscopic data into chemical composition information, wherein the chemometric model is selected in accordance with the item of identification information, wherein, in case of mixtures, the chemometric model for each component is selected, wherein the mixing ratio is used for determining the item of sample information for the mixture
by aggregating the chemometric models depending on the mixing ratio, wherein the aggregating comprises adding and weighing according to the mixing ratio, and/or
as input parameter for at least one forward model.

11. A computer program comprising instructions, wherein when the program is executed by a processing device of the system according to claim 9, the instructions cause the system to perform a method comprising:
i. acquiring spectroscopic data of the sample by using the at least one spectrometer device, within at least one spatial measurement range of the spectrometer device;
ii. acquiring, by using the at least one imaging device, image data of a scene within a field of view of the imaging device, the scene comprising at least a part of the sample and at least a part of the spatial measurement range of the spectrometer device, and analyzing the image data and/or the spectroscopic data thereby determining at least one item of identification information on the sample by using at least one processing device, wherein the image data comprises information about a mixing ratio; and
iii. evaluating the spectroscopic data for obtaining at least one item of sample information by using the processing device, wherein the evaluating comprises processing the spectroscopic data with at least one chemometric model, wherein the chemometric model translates the spectroscopic data into chemical composition information, wherein the chemometric model is selected in accordance with the item of identification information, wherein, in case of mixtures, the chemometric model for each component is selected, wherein the mixing ratio is used for determining the item of sample information for the mixture
by aggregating the chemometric models depending on the mixing ratio, wherein the aggregating comprises adding and weighing according to the mixing ratio, and/or
as input parameter for at least one forward model.

12. A computer-readable storage medium comprising instructions wherein when a program of the computer-readable storage medium is executed by a processing device of the system according to claim 9, the instructions cause the system to perform a method comprising:
i. acquiring spectroscopic data of the sample by using the at least one spectrometer device, within at least one spatial measurement range of the spectrometer device;
ii. acquiring, by using the at least one imaging device, image data of a scene within a field of view of the imaging device, the scene comprising at least a part of the sample and at least a part of the spatial measurement range of the spectrometer device, and analyzing the image data and/or the spectroscopic data thereby determining at least one item of identification information on the sample by using at least one processing device, wherein the image data comprises information about a mixing ratio; and
iii. evaluating the spectroscopic data for obtaining at least one item of sample information by using the processing device, wherein the evaluating comprises processing the spectroscopic data with at least one chemometric model, wherein the chemometric model translates the spectroscopic data into chemical composition information, wherein the chemometric model is selected in accordance with the item of identification information, wherein, in case of mixtures, the chemometric model for each component is selected, wherein the mixing ratio is used for determining the item of sample information for the mixture
by aggregating the chemometric models depending on the mixing ratio, wherein the aggregating comprises adding and weighing according to the mixing ratio, and/or
as input parameter for at least one forward model.

13. A non-transient computer-readable medium including instructions that, when executed by one or more processors, cause the one or more processors to perform the method according to claim 1.

14. A computer program comprising instructions, wherein when the program is executed by a processing device of a system, the instructions cause the system to perform the method according to claim 1.

15. A computer-readable storage medium comprising instructions, wherein when a program of the computer-readable storage medium is executed by a processing device of a system, the instructions cause the system to perform the method according to claim 1.
